(19) United States
(12) Patent Application Publication
(10) Pub. No.: US 2025/0312605 A1
(43) Pub. Date: Oct. 9, 2025

(54) APPARATUS AND METHOD FOR DETERMINING STIMULATION PARAMETERS FOR DEEP BRAIN STIMULATION (DBS)

(71) Applicant: UNIVERSITY OF FLORIDA RESEARCH FOUNDATION, INCORPORATED, Gainesville, FL (US)

(72) Inventors: Jackson N. CAGLE, Gainesville, FL (US); Coralie DE HEMPTINNE, Gainesville, FL (US)

(21) Appl. No.: 18/865,936

(22) PCT Filed: May 25, 2023
(86) PCT No.: PCT/US2023/023543

§ 371 Date: Nov. 14, 2024

Related U.S. Application Data
(60) Provisional application No. 63/345,644, filed on May 25, 2022.

(51) Int. Cl. A61N 1/36; A61N 1/02; A61N 1/05; G16H 20/40

(52) U.S. Cl. CPC A61N 1/36171; A61N 1/025; A61N 1/0534; A61N 1/36185; G16H 20/40

(57) ABSTRACT

A method and apparatus are provided for determining stimulation parameters for DBS. The method includes receiving first data indicating a value of a local field potential (LFP) over a first time period from first contacts positioned adjacent a brain region. The method further includes receiving third data indicating a value of a LFP over second time periods from the first contacts, where the brain region is stimulated by second contacts over second time periods based on stimulation parameter values. The method further includes determining a frequency band encompassing a difference between a first frequency spectrum of the first data and a second frequency spectrum of the third data. The method further includes determining a value of the second frequency spectrum over the frequency band for each stimulation parameter value. The method further includes determining a value of an optimal stimulation parameter based on the value of the second frequency spectrum and each stimulation parameter value.

### CROSS REFERENCE TO RELATED APPLICATIONS

This application claims benefit of U.S. Provisional Application No. 63/345,644, filed May 25, 2022, under 35 U.S.C. § 119(e) and 35 U.S.C. § 120.

### BACKGROUND

Deep brain stimulation (DBS) is an invasive neurosurgical therapy which can be applied for select movement and neuropsychiatric disorders. The classical procedure consists of implanting electrodes in the brain and delivering continuously electrical stimulation through an implanted battery source referred to as an impulse generator.

### SUMMARY

Techniques are provided for determining stimulation parameters for deep brain stimulation (DBS).

The inventors of the present invention recognized that with conventional methods for performing DBS, after a patient is implanted with DBS electrode(s) and stimulator(s), the stimulation is turned on. However, selecting the optimal stimulation parameters is a manual process done by a movement disorders specialist that is often laborious and time consuming. It consists in adjusting one or more of the contact, the amplitude, the pulse width, and the frequency of the stimulation over long periods of time in clinic visits (e.g., typically several monthly visits for 6 months). During this trial-and-error process, clinicians test many stimulation parameters while visually assessing the benefit of stimulation on patient symptoms severity (such as tremor), as well as the side effects (such as muscle pulling).

As shown in FIGS. 1C and 1D, the target of stimulation in these conventional methods includes brain regions, such as the subthalamic nucleus (STN) region 120 (FIG. 1C) and/or the globus pallidus internus (GPi) region 122 (FIG. 1D). However, a subregion 121, 123 within each respective brain region 120, 122 is the actual target that needs to be stimulated in order to achieve therapeutic benefit. The inventors recognized that although these brain regions 120, 122 are visible on brain imaging as shown in FIGS. 1C and 1D, the respective subregions 121, 123 that need to be stimulated to improve patient's symptoms are often not easily identifiable on imaging and is patient-specific (different in each patient). Thus, although FIGS. 1C and 1D label the therapeutic subregions 121, 123 these regions are labeled for illustrative purposes only, as they are not easily identifiable with imaging systems.

FIGS. 1E and 1F depict an example of contacts 110a, 110b that are used to stimulate the region. These contacts 110a, 110b are non-insulated portions of a lead 105 that electrically connect the contacts 110a, 110b to a stimulation source (e.g. impulse generator 102 and/or controller 104 in FIG. 1A). FIG. 1E depicts a ring contact 110a that encircles the lead 105 and is configured to symmetrically stimulate the brain region around a circumference of the lead 105. FIG. 1F depicts a segment contact 110b that is positioned along one side of the lead 105 and is configured to stimulate the brain region in a single direction (e.g. along the same side of the lead 105 as the segment contact 110b). Changing the stimulation parameters allows one to ‘shape’ the stimulation toward the therapeutic subregion 121, 123 and to avoid subregions associated with stimulation induced side effects (e.g. portions of the brain region 120, 122 other than the subregions 121, 123). An example of this ‘shaping’ is shown in FIGS. 1E and 1F. When increasing the stimulation (e.g., from 1 milliamp (mA) to 3 mA (dark grey to light grey) the electrical field around the lead 105 gets larger, and thus a larger volume of brain tissue will be activated with larger stimulation amplitude. Selecting the specific contact to deliver stimulation can be used to steer the current toward a specific direction, as shown in FIG. 1F where the segment contact 110b is used to direct stimulation to the left. In one example, the segment contact 110b is activated, and the electrical field is steered toward the left side of the lead 105 (compared to the symmetrical field when stimulation from a ring contact 110a). Therefore, adjusting these parameters help to focus the stimulation to the optimal subregion. The inventors of the present invention recognized that currently, the assessment of therapeutic effect of the stimulation is based on subjective assessment of patient's symptoms by a clinician.

To overcome the above-noted drawbacks of conventional methods to determine stimulation parameters, the inventors of the present invention developed the algorithm discussed herein. The algorithm uses the stimulation induced change in oscillatory brain activity as an objective measure to identify optimal stimulation parameters. The algorithm obtains recordings of local field potentials while stimulating the brain with different stimulation settings and determines therapeutic settings with computation modeling. This method is based on objective stimulation induced changes in brain signals and therefore does not require a movement disorder specialist to subjectively assess the benefit of each stimulation setting. The inventors of the present invention developed this method to reduce the long trial-and-error process of conventional methods to a single recording session during which the brain signal will be recorded, while testing different stimulation settings. The algorithm will then use data from this single recording session to predict the optimal stimulation parameters. The recording session may be done once the contact(s) are placed in the brain and thus could be done during the surgery; after the surgery and before the patient is discharged from the hospital; at a visit in-clinic or even at patient's home (e.g., for devices allowing for remote control). This method significantly reduces the time spent on programming for both the patient and the clinician, in order to achieve better efficacy and less side effects.

In a first set of embodiments, a method is provided for determining stimulation parameters for DBS. The method includes a) receiving, at a processor, first data indicating a value of a local field potential (LFP) over a first time period from one or more first contacts positioned adjacent a region of a brain of a subject. The method also includes b) receiving, at the processor, second data indicating a first frequency spectrum of the first data over the first time period. The method further includes c) receiving, at the processor, third data indicating a value of a LFP over each of a respective plurality of second time periods from the one or more first contacts. The region of the brain is stimulated by one or more second contacts over the respective plurality of second time periods based on a respective plurality of values of a stimulation parameter. The method further includes d) receiving, at the processor, fourth data indicating a value of a plurality of second frequency spectrums of the third data for each of the respective plurality of values of the stimulation parameter. The method further includes e) determining, with the processor, a frequency band that encompasses a characteristic of a difference between a value of a parameter of the first frequency spectrum and a value of a parameter of one of the plurality of second frequency spectrums. The method further includes f) determining, with the processor, a value of a parameter of each of the plurality of second frequency spectrums over the frequency band for each respective value of the stimulation parameter. The method further includes g) determining, with the processor, a value of an optimal stimulation parameter based on the value of the parameter of each of the plurality of second frequency spectrums and each respective value of the stimulation parameter from step f).

In a second set of embodiments, an apparatus is provided that is configured to perform one or more steps of the method of the first set of embodiments.

In a third set of embodiments, a non-transitory computer readable medium carrying one or more sequences of instructions to cause one or more processors to perform one or more steps of the method of the first set of embodiments.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1A is a block diagram that illustrates an example of an apparatus for determining stimulation parameters for DBS, according to one embodiment;

FIG. 1B is an image that illustrates an example of a side perspective view of an electrode and contacts of the apparatus of FIG. 1A, according to one embodiment;

FIGS. 1C and 1D are images that illustrate an example of a cross-sectional view of different regions of the brain to be stimulated with the apparatus of FIG. 1A, according to one embodiment;

FIGS. 1E and 1F are images that illustrate an example of a side view of the contacts of FIG. 1B, according to one embodiment;

FIGS. 2A and 2B are images that illustrate an example of a top sectional view of contacts of the apparatus of FIG. 1A implanted adjacent a region of a brain, according to one embodiment;

FIGS. 2C and 2D are images that illustrate an example of a side sectional view of contacts of the apparatus of FIG. 1A implanted adjacent a region of a brain, according to one embodiment;

FIGS. 3A and 3B are graphs that illustrate an example of a stimulation signal used to cause one or more contacts of FIG. 1A to stimulate the region of the brain over a plurality of time periods, according to one embodiment;

FIG. 3C is a graph that illustrates an example of a neural signal measured by one or more contacts of FIG. 1A over the plurality of time periods, according to one embodiment;

FIG. 3D is a graph that illustrates an example of a Fourier transform of the neural signal of FIG. 3C over a time window, according to one embodiment;

FIG. 3E is an image that illustrates an example of a spectrogram of the neural signal of FIG. 3C over the plurality of time periods, according to one embodiment;

FIG. 3F is an image that illustrates an example of a spectrogram of a neural signal based on the stimulation signal of FIG. 3G, according to one embodiment;

FIG. 3G illustrates an example of a stimulation signal used to cause one or more contacts of FIG. 1A to stimulate the region of the brain over a plurality of time periods, according to one embodiment;

FIG. 3H is an image that illustrates an example of a spectrogram of a neural signal based on the stimulation signal of FIG. 3I, according to one embodiment;

FIG. 3I illustrates an example of a stimulation signal used to cause one or more contacts of FIG. 1A to stimulate the region of the brain over a plurality of time periods, according to one embodiment; FIG. 4A is a graph that illustrates an example of a power spectrum indicating an average amplitude at each frequency using different contacts without stimulation, according to one embodiment;

FIG. 4B is a graph that illustrates an example of a power spectrum indicating an average amplitude at each frequency using a first contact based on the stimulation signal of FIGS. 3A and 3B at different amplitudes, according to one embodiment;

FIG. 4C is a graph that illustrates an example of a power spectrum indicating an average amplitude at each frequency using a second contact based on the stimulation signal of FIGS. 3A and 3B at different amplitudes, according to one embodiment;

FIGS. 4D and 4E are graphs that illustrate an example of a change in the power spectrum indicating an average amplitude at each frequency using first and second contacts based on the stimulation signal of FIGS. 3G and 3I at different amplitudes, according to one embodiment;

FIG. 5 is a graph that illustrates a best fit curve of an average amplitude of the power spectrum of FIG. 4B over a frequency band for each stimulation amplitude, according to one embodiment;

FIG. 6 is a graph that illustrates an example of a best fit curve of an average amplitude of the power spectrum of FIG. 4C over a frequency band for each stimulation amplitude, according to one embodiment;

FIG. 7 is a flow diagram that illustrates an example of a method for determining stimulation parameters for deep brain stimulation, according to one embodiment;

FIGS. 8A and 8B are box violin plots that illustrate an example of a correlation between average beta power and bradykinesia score, according to one embodiment;

FIGS. 8C and 8D are plots that illustrate an example of a correlation between average beta power and bradykinesia score, according to one embodiment;

FIG. 9 is a block diagram that illustrates a computer system upon which an embodiment of the invention may be implemented;

FIG. 10 is a block diagram that illustrates a chip set upon which an embodiment of the invention may be implemented; and

FIG. 11 is a block diagram that illustrates a mobile terminal upon which an embodiment of the invention may be implemented.

### DETAILED DESCRIPTION

A method and apparatus are described for determining stimulation parameters for deep brain stimulation (DBS). In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Some embodiments of the invention are described below in the context of determining stimulation parameters for deep brain stimulation (DBS) of the globus pallidus internus (GPi) to reduce symptoms of Parkinson's Disease (PD). However, the invention is not limited to this context. In other embodiments, the embodiments of the invention can be used for determining stimulation parameters for DBS in other regions of the brain, such as subthalamic nucleus (STN), thalamus, globus pallidus internus (GPi). In still other embodiments, the embodiments of the invention can be used to reduce symptoms of other conditions, such as essential tremor to stimulate the thalamus (ventralis intermediate nucleus (Vim) of the thalamus).

It has been established that there is a correlation between symptoms of neurological conditions (e.g. PD) and an amplitude of a power spectrum of signals from a brain region [1], [2]. Thus, stimulated-induced changes in the signals from the brain region can be used to reduce the power spectrum and thus alleviate symptoms of these neurological conditions. The inventors of the present invention recognized while this correlation between PD symptoms and power spectrum was known, no algorithm has been established that can predict or determine values of one or more stimulation parameters in order to achieve a reduction in the power spectrum and consequently a reduction in symptoms of these neurological conditions. Instead, conventional methods merely use a trial-and-error approach where clinicians randomly guess different stimulation parameters and clinically observe whether these random stimulation parameters have a therapeutic effect.

### 1. Overview

Deep brain stimulation (DBS) involves implanting electrodes within certain areas of the brain. These electrodes produce electrical impulses that regulate abnormal impulses or can affect certain cells and chemicals within the brain. The amount of stimulation in deep brain stimulation is controlled by a pacemaker-like device placed under the skin in the upper chest. A wire that travels under the skin connects this device to the electrodes in the brain. Deep brain stimulation is commonly used to treat a number of conditions, such as: Parkinson's disease, Essential tremor, Dystonia, Epilepsy and Obsessive-compulsive disorder. Deep brain stimulation is also being studied as a potential treatment for: Tourette syndrome, Huntington's disease and chorea, Chronic pain and Cluster headache.

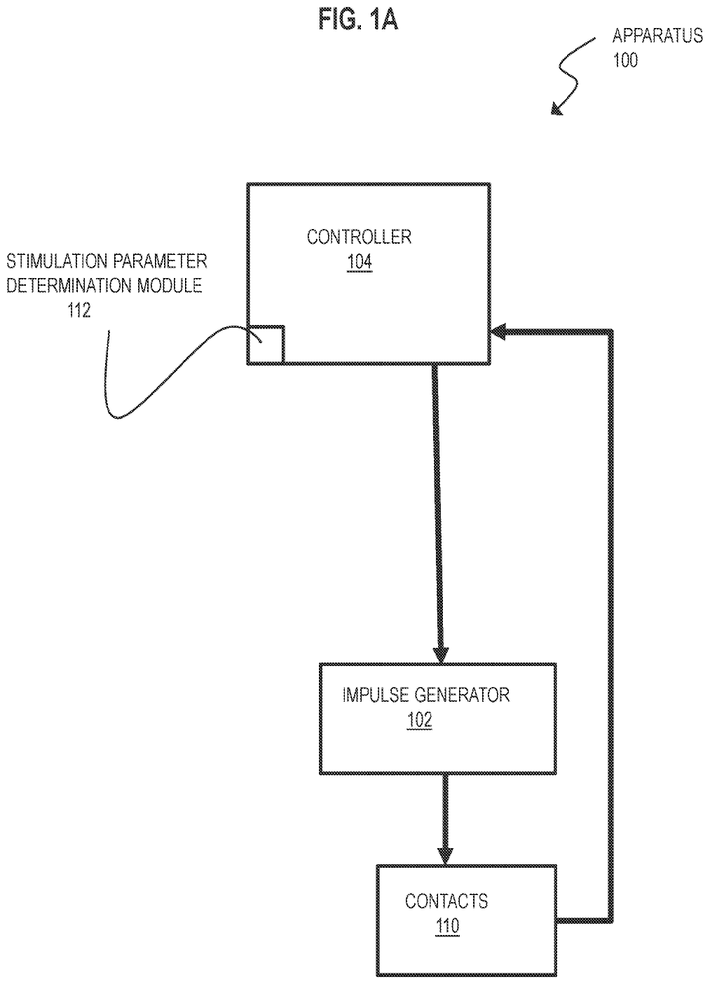

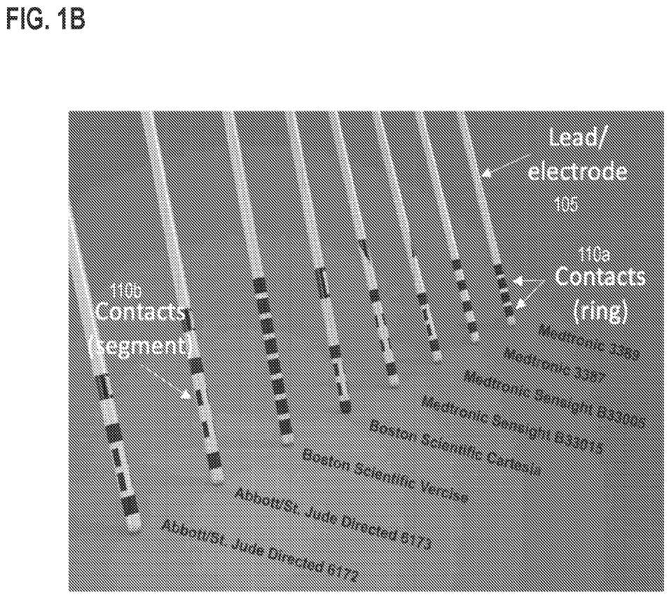

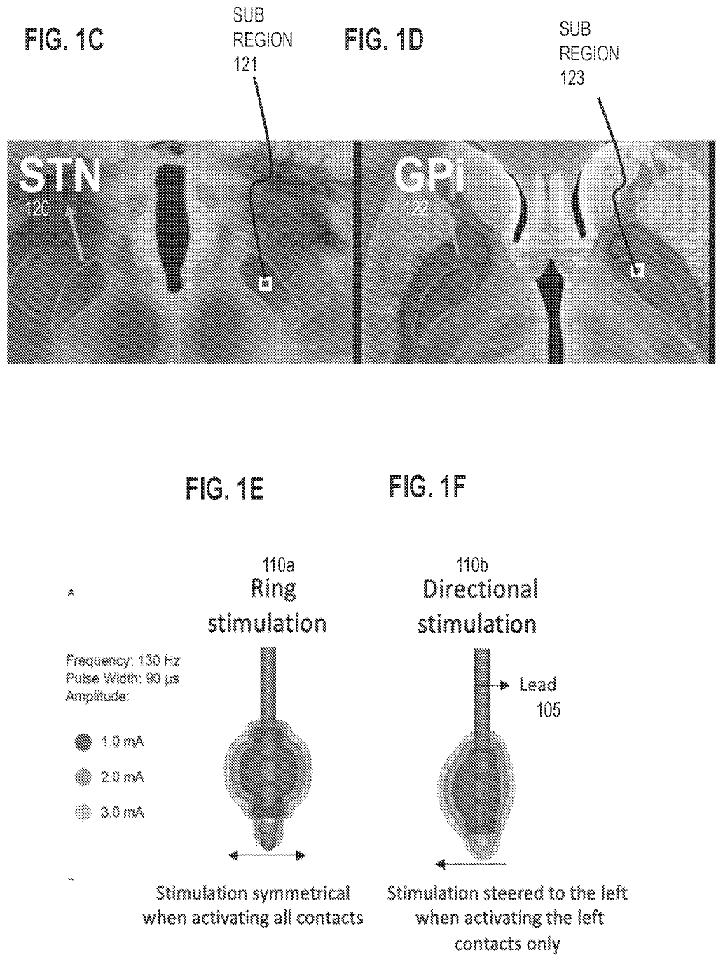

FIG. 1A is a block diagram that illustrates an example of an apparatus 100 for determining stimulation parameters for DBS, according to one embodiment. In an embodiment, the apparatus 100 includes contacts 110 that are configured to be implanted in a head of a subject (e.g. adjacent a region of the brain). In an embodiment, the apparatus 100 also includes an impulse generator 102 that is also configured to be implanted in the head of the subject. In one embodiment, the impulse generator 102 is configured to transmit a signal to one or more of the contacts 110 to cause the one or more contacts 110 to stimulate a region of the brain. In some embodiments, as shown in FIG. 1B the contacts 110 are provided along a respective electrode or lead 105. In these embodiments, the contacts 110 are non-insulated portions of the lead 105. In an example embodiment, the contacts 110 include one or more ring contacts 110a (FIG. 1E) and/or one or more segment contacts 110b (FIG. 1F). In this example embodiment, the ring contacts 110a are 360-degree contacts which are configured to stimulate the brain region in all directions within 360-degrees of the lead 105. In this example embodiment, the segment contacts 110b include a ring contact divided into three or more segments (e.g. which sum up to 360 degrees) and thus are configured to stimulate the brain region within a limited angular range of the lead 105 (e.g. 120 degrees for those segment contacts based on a ring contact divided into three segments). As shown in FIG. 1B, in some embodiments the contacts 110 are spaced apart by varied spacing for different leads 105 or for different regions along the lead 105.

As previously discussed, these contacts 110a, 110b stimulate the brain region with a different distribution (different direction) and thus can be selectively used depending on the location of the sub region 121, 123 (FIGS. 1C and 1D) that needs to be stimulated. In one example embodiment, if the contact 110 is positioned within the sub region 121, 123 then the ring contact 110a could be used, to evenly stimulate the sub region 121, 123 around a circumference of the ring contact 110b. In another example embodiment, if the contact 110 is positioned to one side of the sub region 121, 123 then the segment contact 110b could be used that directs stimulation in the direction of the sub region 121, 123 and does not direct stimulation in other directions, so to minimize side effects due to stimulation other regions beyond the sub region 121, 123. Although ring contacts and segment contacts are discussed herein, the contacts 110 are not limited to these particular contacts and include any contact design appreciated by one of ordinary skill in the art.

In one embodiment, the apparatus 100 also includes a controller 104 that is communicatively coupled to the impulse generator 102. In an example embodiment, the controller 104 is configured to transmit one or more signals to the impulse generator 102 which in turn causes the impulse generator 102 to transmit one or more signals to the contacts 110 to stimulate the region of the brain (e.g., sub region 121, 123). In an example embodiments, a value of one or more parameters of the signal(s) sent from the controller 104 and/or from the impulse generator 102 can be changed, which in turns changes the parameters of stimulation of the region of the brain.

In one embodiment, the contacts 110 have an outlet that is communicatively coupled to an inlet of the controller 104. In this embodiment, one or more contacts 110 (e.g. cathode that are positively charged) measures a value of a signal (e.g. local field potential (LFP)) as the region of the brain is being stimulated by other contacts 110 (e.g. anode that are negatively charged). In an example embodiment, the one or more contacts 110 that measure the value of the signal transmit data to the controller 104 that indicates the value of these signals during the time period that the region of the brain is being stimulated. In some embodiments, some of the contacts 110 (e.g. anodes) are communicatively coupled to the impulse generator 102 (e.g. to receive signals to stimulate the region of the brain) and others (e.g. cathodes) are connected to the controller 104 (e.g. to transmit data indicating parameter values of measured signals during the stimulation of the brain region).

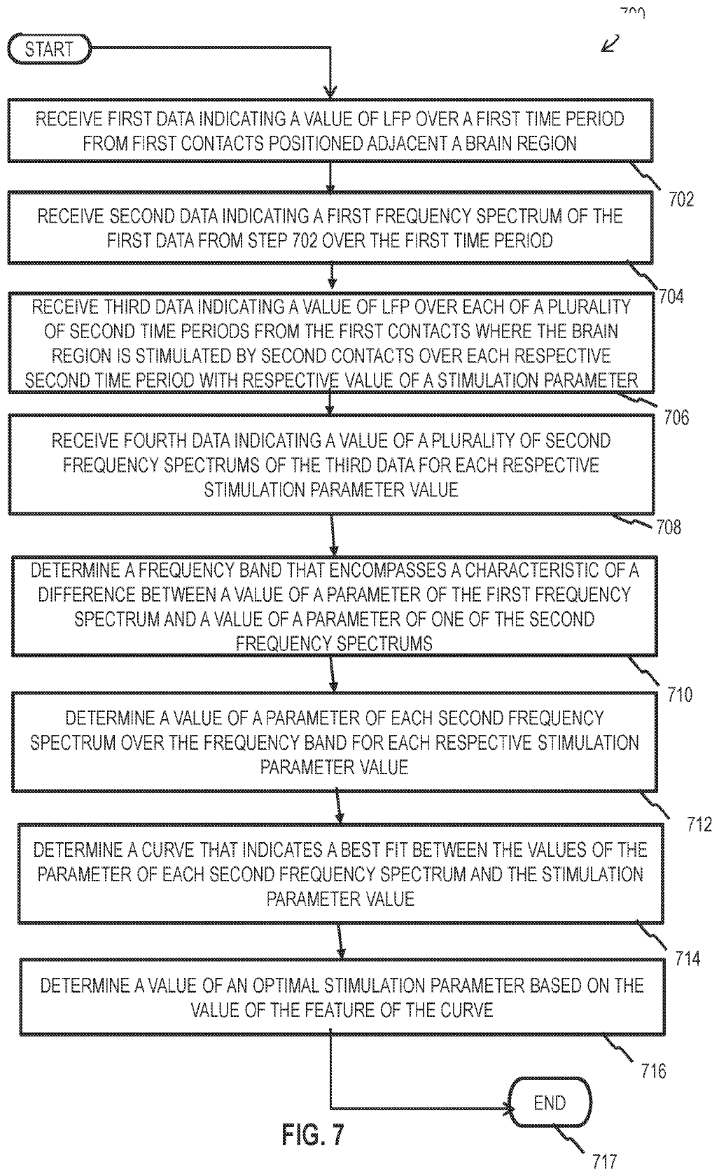

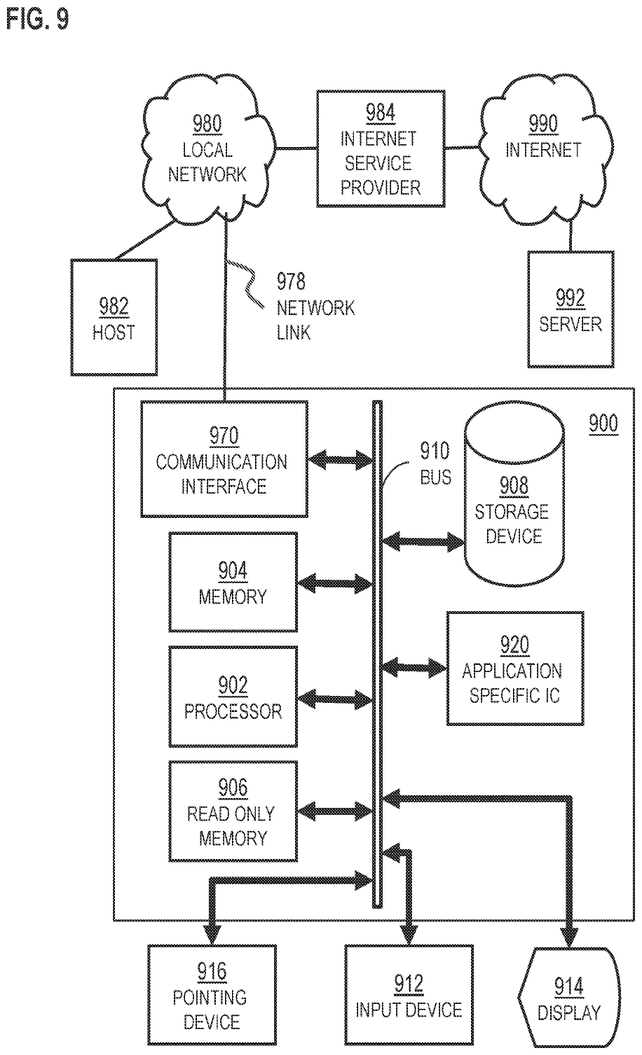

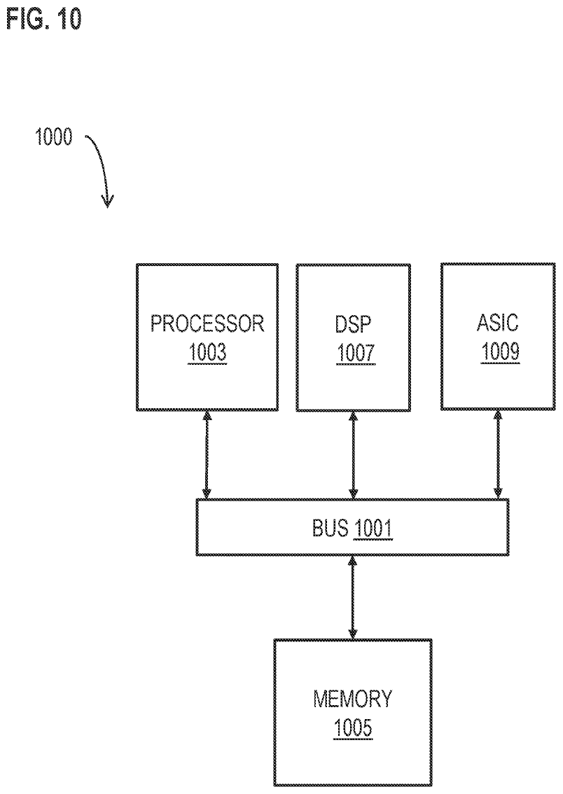

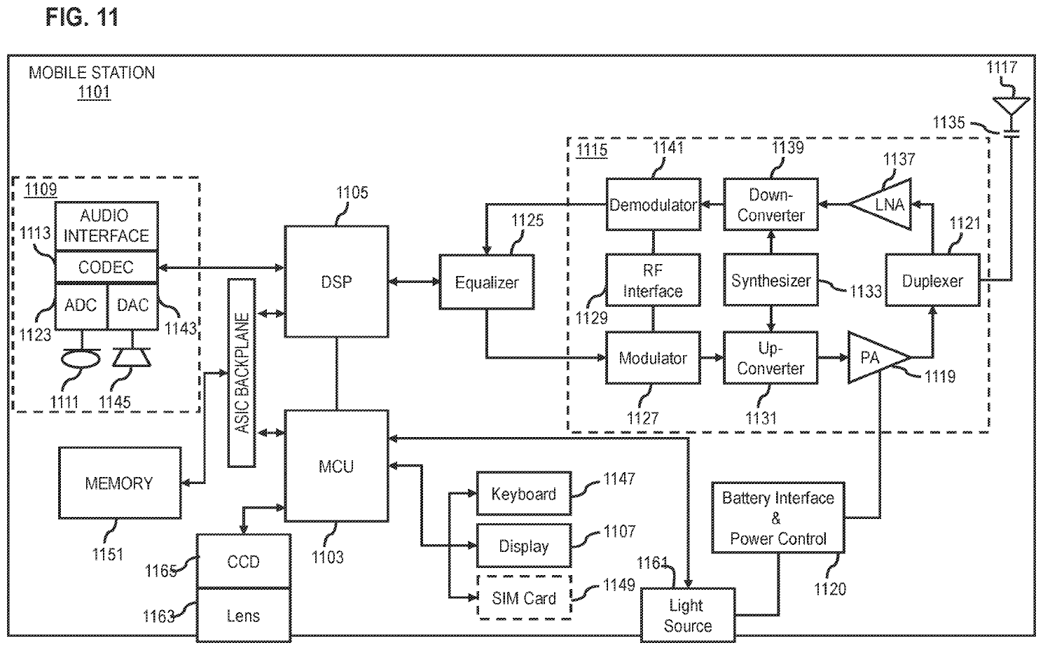

The controller 104 includes a module 112 to determine values of one or more stimulation parameters of the contacts 110. In an embodiment, the module 112 includes or more sequences of instructions to cause the controller 104 to perform one or more steps of the method 700 discussed with respect to the flowchart of FIG. 7. In some embodiments, the module 112 causes the controller 104 to perform one or more steps to determine or select values of stimulation parameters (e.g. amplitude, frequency, pulse width, etc.) of the contacts 110 to stimulate the region of the brain such that symptoms of one or more conditions (e.g. Parkinson's Disease) are reduced. In other embodiments, the module 112 causes the controller 104 to perform one or more steps to determine or select values of other parameters (e.g. a selection of one or more optimal contacts 110 among the plurality of contacts 110 to use to perform the stimulation, a selection of a type of contact (e.g. ring contact or segment contact) to use to perform the stimulation, etc.) to stimulate the region of the brain so to alleviate or reduce the symptoms of the condition (e.g. Parkinson's Disease). In some embodiments, the processor or controller 104 is a computer system as described below with reference to FIG. 9, a chip set described below with reference to FIG. 10 or a mobile terminal described below with reference to FIG. 11.

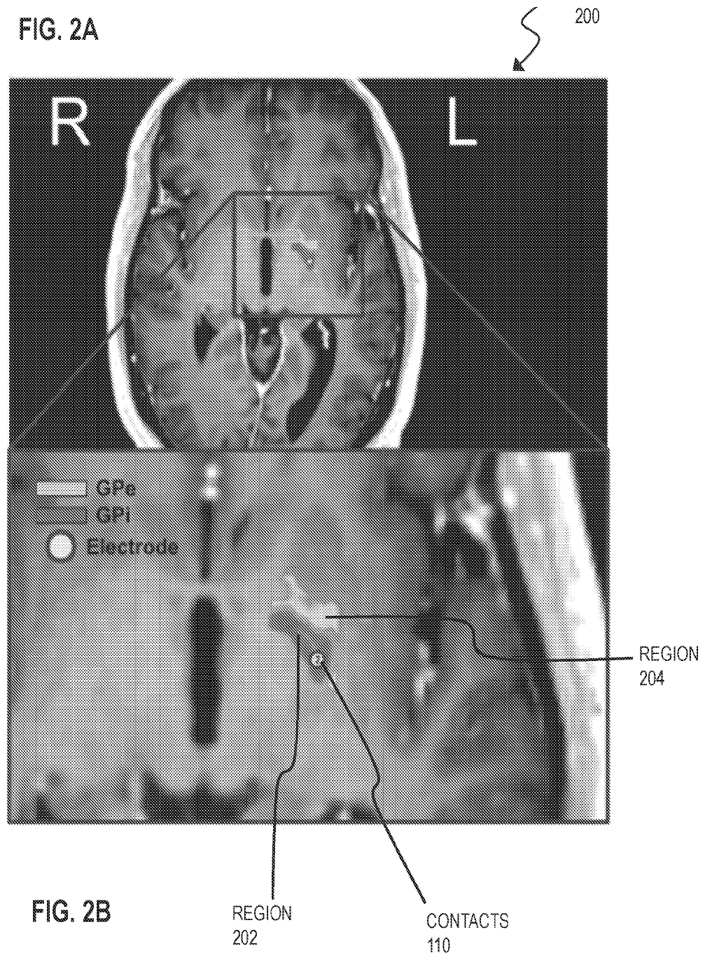

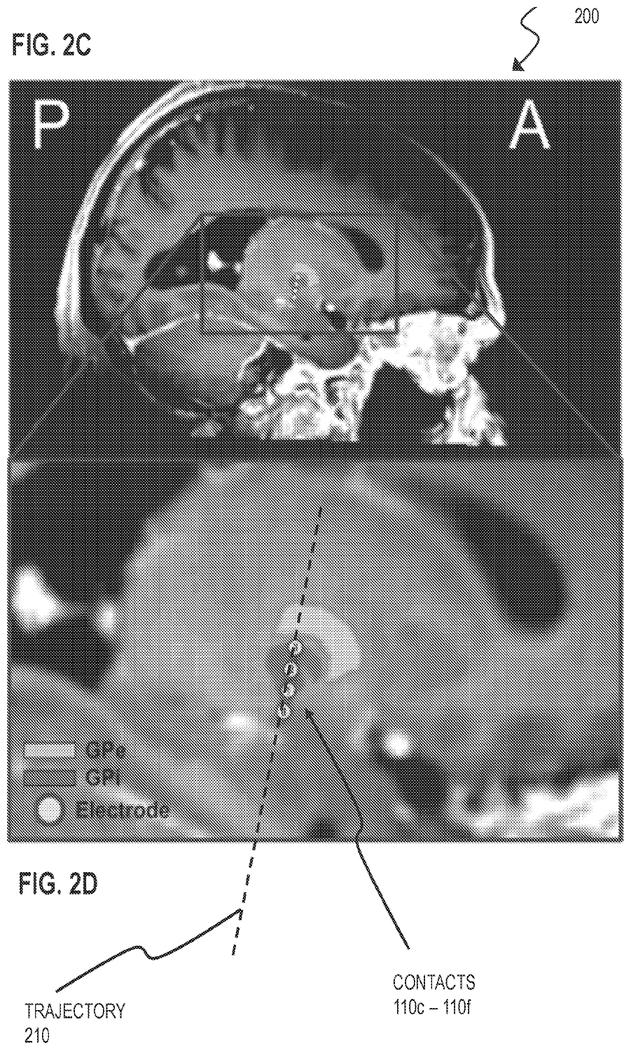

The placement of the contacts 110 adjacent the region of the brain of the subject will now be discussed. In some embodiments, the method discussed herein does not include the step of surgically implanting the contacts 110 adjacent the region of the subject. However, in some embodiments, the method discussed herein may include one or more data gathering steps performed during and/or after this surgery (e.g. measuring signal parameters given off by the brain region during and/or after this surgery with no stimulation and with stimulation with various stimulation parameter values). FIGS. 2A and 2B are images that illustrate an example of a top sectional view of contacts 110 of the apparatus 100 of FIG. 1 implanted adjacent a region 202 of a brain, according to one embodiment. In one embodiment, the region 202 is the globus pallidus internus (GPi). In other embodiments, the electrodes 110 are implanted in a different region 204 of the brain (e.g., tripeptide glycine-proline-glutamate (GPe)). FIG. 2B shows an enlargement of the region 202 of the brain in FIG. 2A and depicts that the contacts 110 are placed within the region 202 such that they are approximately stacked vertically above one another. However, in other embodiments, the contacts 110 can be positioned in a different trajectory within this top sectional view of the brain (e.g. can be positioned such that they are not vertically stacked relative to one another).

FIGS. 2C and 2D are images that illustrate an example of a side sectional view of contacts 110 of the apparatus 100 of FIG. 1A implanted adjacent the region 202 of the brain, according to one embodiment. In an embodiment, the contacts 110 are implanted in the region 202 of the brain along a trajectory 210. The trajectory 210 can be selected prior to surgically implanting the contacts 110 in order to achieve a desired distribution of the contacts 110 within the region 202 (e.g. such that each contact 110 is arranged in the region 202 and/or are arranged in a particular configuration relative to each other, e.g. linear arrangement). As shown in FIG. 2D the contacts 110 includes a plurality of contacts 110c, 110d, 110e, 110f (also referred to respectively as contacts 0-3 or E00-E03 herein) that have different anterior-posterior placement relative to each other. In this example embodiment, contact 110c (contact 0 or E00) is the most posterior placed contact, whereas contact 110f (contact 3 or E03) is the most anterior placed electrode.

In an embodiment, when using the apparatus 100 one of the contacts 110 stimulates the region of the brain while one or more non-adjacent contacts 110 (e.g. pair of non-adjacent contacts) to the stimulating contact 110 are used to measure a neural signal from the brain region (e.g. LFP). In one example embodiment, when the contact 1 (E01) stimulates the brain region, the non-adjacent contact pair 0-2 (E00-E02) measure the neural signal. In another example embodiment, when the contact 2 (E02) stimulates the brain region, the non-adjacent contact pair 1-3 (E01-E03) measure the neural signal.

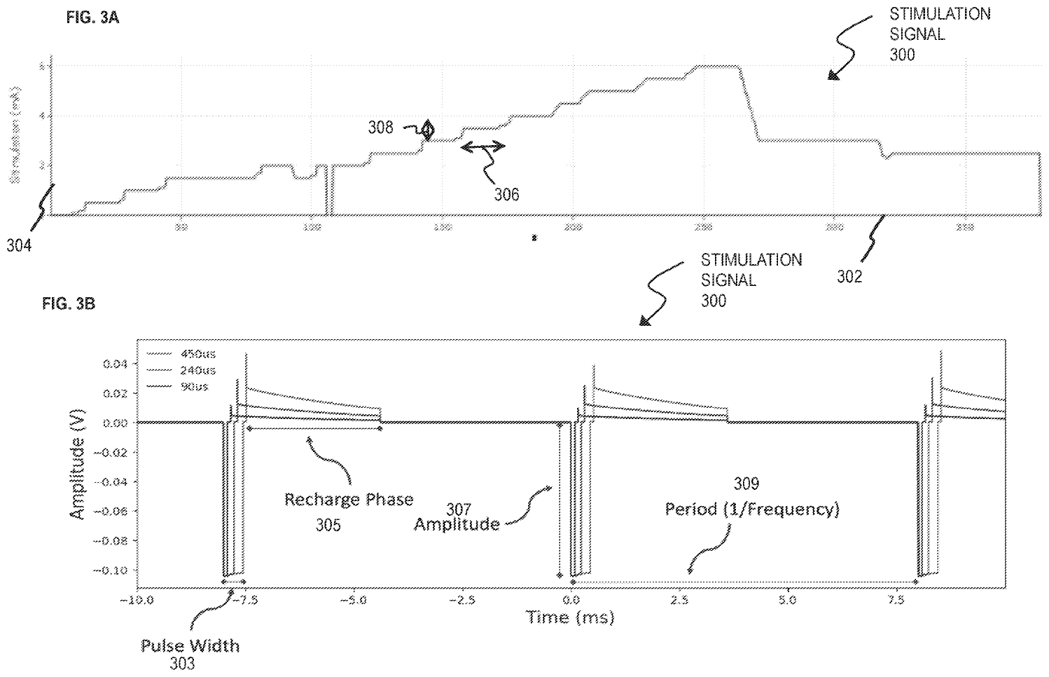

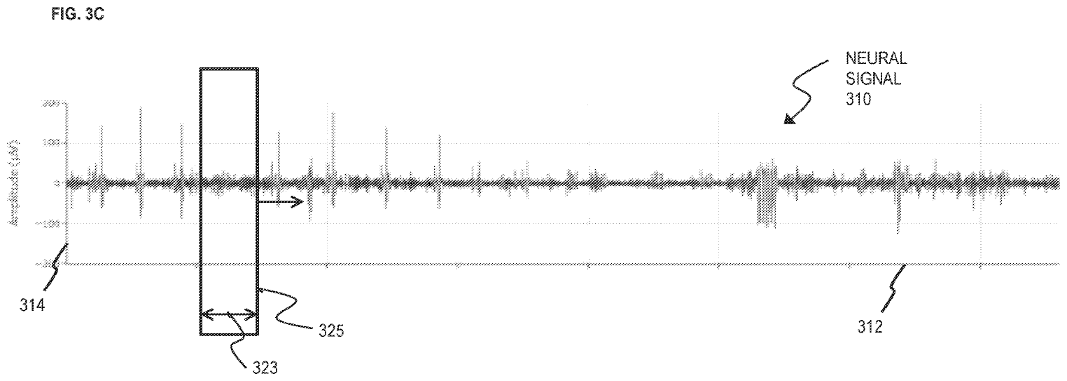

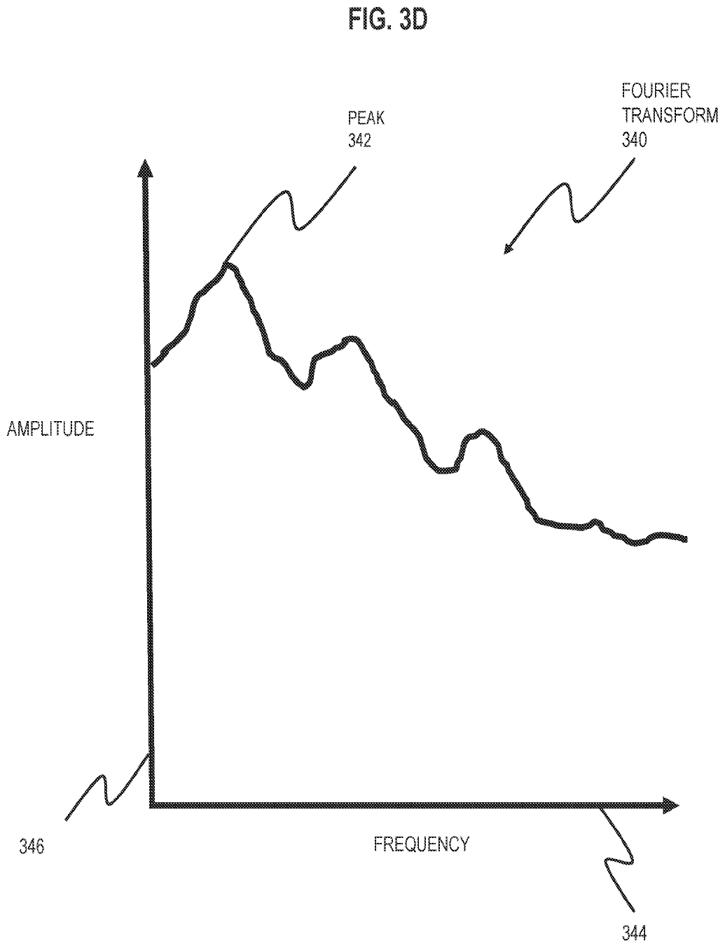

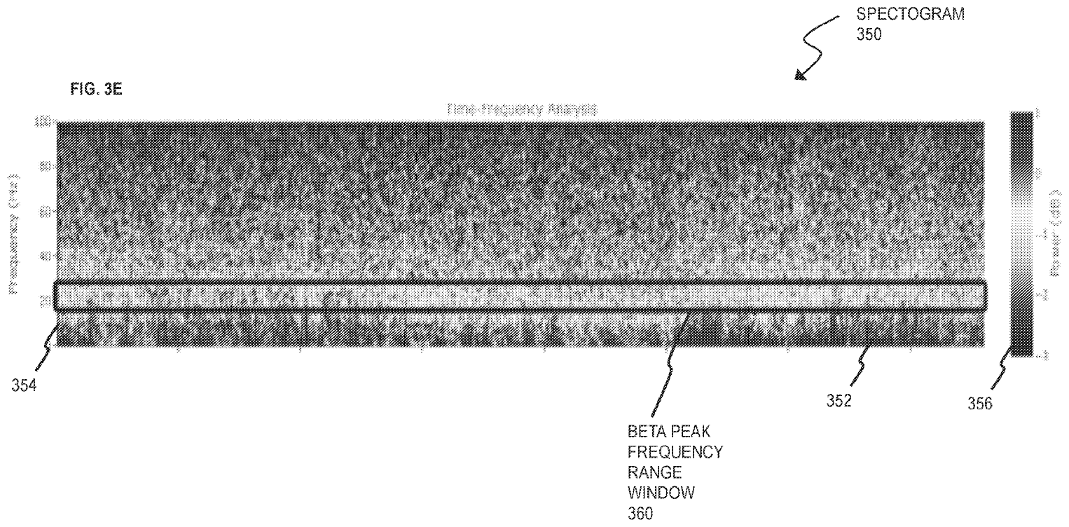

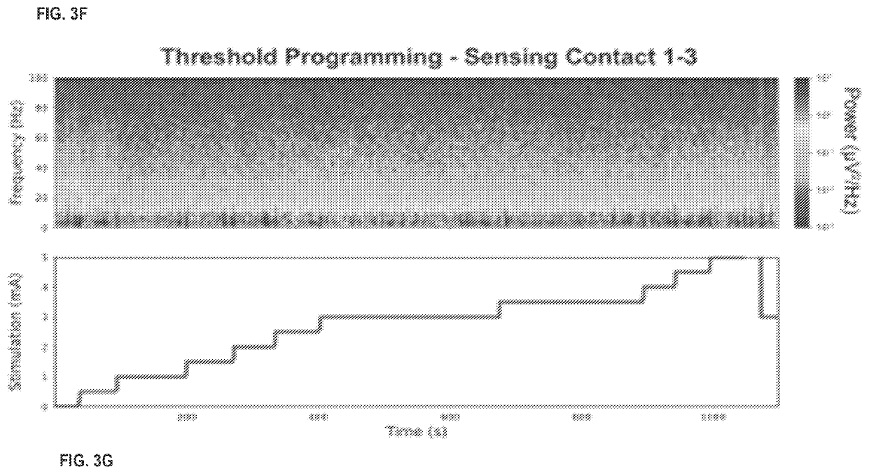

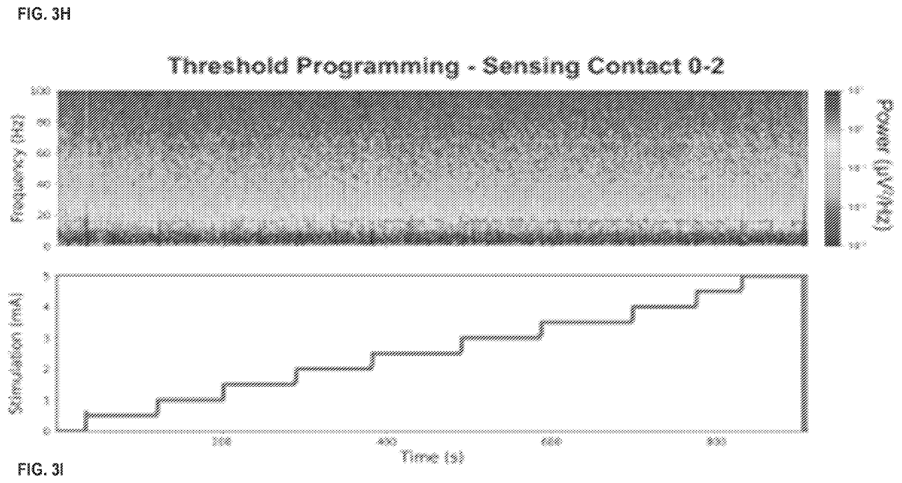

The stimulation of the brain region using the contacts 110 will now be discussed. In an embodiment, a signal is transmitted from the impulse generator 102 to one or more contacts 110 to stimulate the brain region (e.g. GPi). FIG. 3A is a graph that illustrates an example of a stimulation signal 300 used to cause one or more contacts 110 of FIGS. 2C and 2D to stimulate the region of the brain over a plurality of time periods, according to one embodiment. In one embodiment, the stimulation signal 300 is a stepwise function. The horizontal axis 302 is time in units of seconds. The vertical axis 304 is current in units of milliamps (mA). In an embodiment, this signal is generated by the controller 104 and/or the impulse generator 102 and transmitted to the contacts 110. In one example embodiment, the stimulation signal 300 is transmitted to contact 1 (E01). In another example embodiment, the stimulation signal 300 is transmitted to contact 2 (E02). As shown in FIG. 3A, the stimulation signal 300 is divided into multiple time periods 306, where the amplitude of the signal 300 increases by an incremental amount 308 for each time period. In an example embodiment, the incremental amount 308 is about 0.5 mA or in a range from about 0.25 mA to about 0.75 mA. In another example embodiment, the time period 306 is about 20 seconds or in a range from about 15 seconds to about 25 seconds. In an example embodiment, one or more other parameters (e.g. pulse width, frequency) of the stimulation signal 300 are fixed during the stimulation of the brain region. However, in other embodiments, these one or more other parameters can be varied for each time period 306 while the amplitude of the stimulation signal 300 is fixed at each time period 306.

FIG. 3B is a graph that illustrate an example of the stimulation signal 300 within each time period 306. In this embodiment, the graph of FIG. 3B shows multiple parameters of the stimulation signal 300 that can be varied between each time period 306. In one embodiment, the amplitude 307 is varied between each time period 306 (e.g. by the incremental amount 308 in FIG. 3A) while the remaining parameters including pulse width 303, recharge phase 305 and frequency 309 are held fixed between each time period 306. However, in other embodiments one of the pulse width 303, recharge phase 305 and frequency 309 are varied between each time period 306. In an embodiment, the algorithm disclosed herein is used to select an optimal stimulation parameter over each time period 306 to enhance the therapeutic benefit to the subject. In these embodiments, values of any of the pulse width 303, recharge phase 305, amplitude 307 and frequency 309 can be selected with the algorithm disclosed herein, in order to enhance the therapeutic benefit to the subject (e.g. reduced symptoms of PD).

The neural signal detected from the brain region using the contacts 110 during the stimulation will now be discussed. FIG. 3C is a graph that illustrates an example of a neural signal 310 measured by one or more contacts 110 of FIGS. 2C and 2D over the plurality of time periods 306, according to one embodiment. The horizontal axis 312 is time in units of seconds. The vertical axis 314 is amplitude in arbitrary units. In an embodiment, the neural signal 310 is detected by a non-adjacent pair of contacts 110. The contacts 110 transmit data to the controller 104 that indicates the values of the neural signal 310 over each time period 306

Frequency characteristics are now obtained of the neural signal 310, which are then used to determine the stimulation parameters of DBS. The controller 104 receives data from the contacts 110 indicating the values of the neural signal 310 and subsequently determines frequency characteristics of the neural signal 310. In one embodiment, these frequency characteristics include a frequency spectrum or Fourier transform of the neural signal 310 values. FIG. 3D is a graph that illustrates an example of a Fourier transform 340 of the neural signal 310 of FIG. 3C over a time window, according to one embodiment. The horizontal axis 344 is frequency in arbitrary units. The vertical axis 346 is amplitude in arbitrary units. FIG. 3C depicts a time window 325 having a width 323 along the neural signal 310. In an example embodiment, the time window width 323 is about 1 second or in a range from about 0.5 seconds to 1.5 seconds. The Fourier transform 340 of FIG. 3D depicts the frequency spectrum of the neural signal 310 over the time window 325. In one embodiment, the value of the width 323 is selected such that the frequency characteristics of the neural signal 310 is relatively constant over the width 323. The peak 342 of the Fourier transform 340 in FIG. 3D indicates a maximum amplitude of a peak frequency over the time window 325. In this example embodiment, the peak 342 of FIG. 3D corresponds to a frequency value along axis 344 which has a maximum contribution to the neural signal time window 325 relative to other frequencies.

In an embodiment, as the time window 325 moves along the time axis 312 of the neural signal 310, a Fourier transform 340 such as depicted in FIG. 3D is generated for each respective time window 325. In an example embodiment, the time window 325 is moved by an increment that is less than the width 323 so that there is some overlap (e.g. about 0.5 seconds) between the time windows 325. In one example embodiment, the overlap is about half of the width 323. Thus, in some embodiments the Fourier transform 340 is generated over a plurality of overlapping time windows 325. However, in other embodiments, the time windows 325 are non-overlapping such that the Fourier transforms 340 are not generated over overlapping time windows 325.

The multiple Fourier transforms 340 that are generated over the plurality of time windows 325 are then combined into a spectrogram. The spectrogram simultaneously depicts the Fourier transforms 340 for each time window 325 over the plurality of time periods 306 of the stimulation signal 310. FIG. 3E is an image that illustrates an example of a spectrogram 350 of the neural signal 310 of FIG. 3C over the plurality of time periods 306, according to one embodiment. The horizontal axis 352 is time in units of seconds. The vertical axis 354 is frequency in units of Hertz (Hz). Grey scale 356 is used to show the units of power (dB) at each time and frequency value. In an embodiment, each vertical slice of the spectrogram 350 corresponds to the Fourier transform 340 over a respective time window 325 along the plurality of time periods 306.

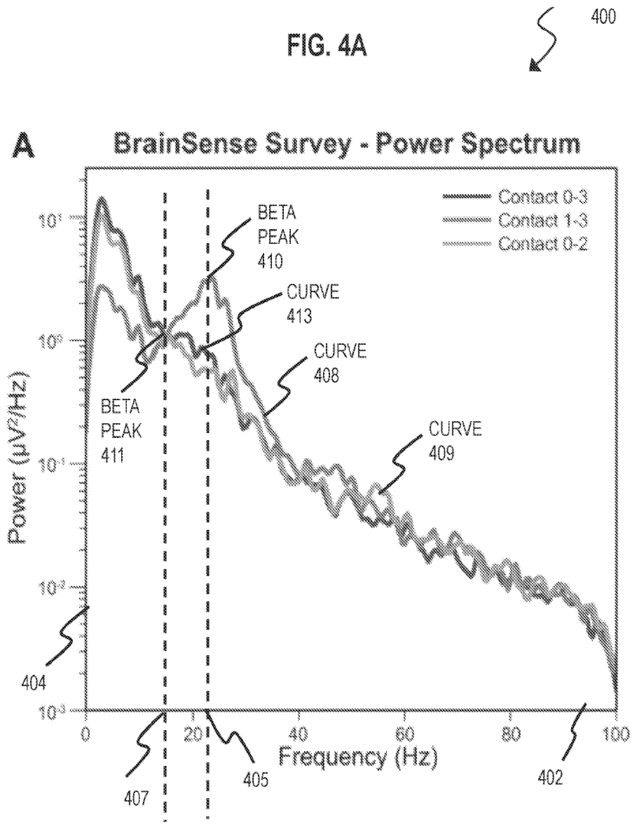

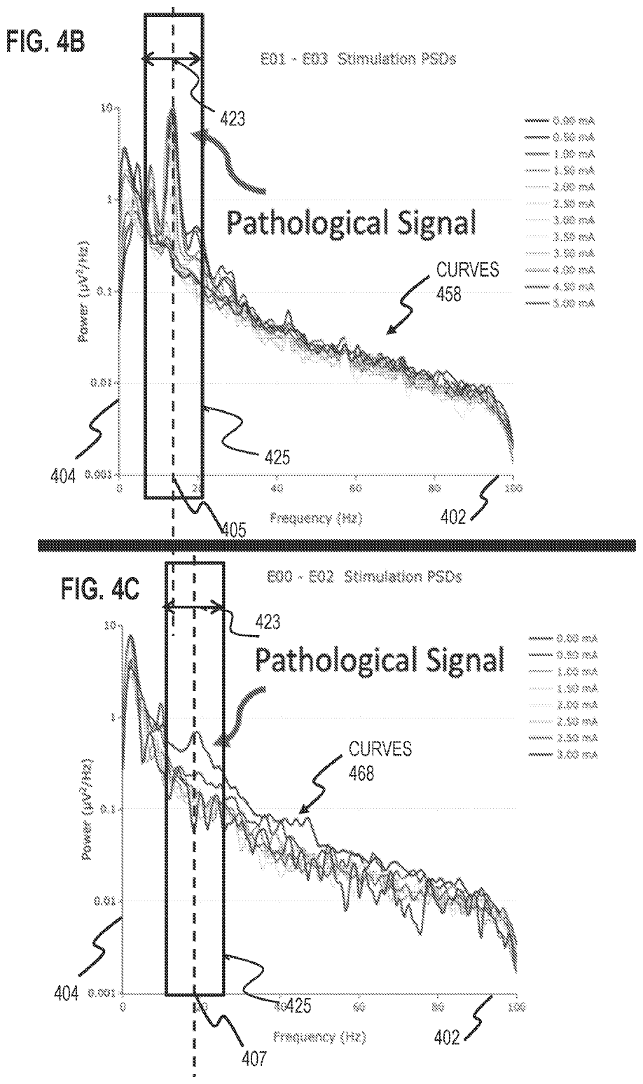

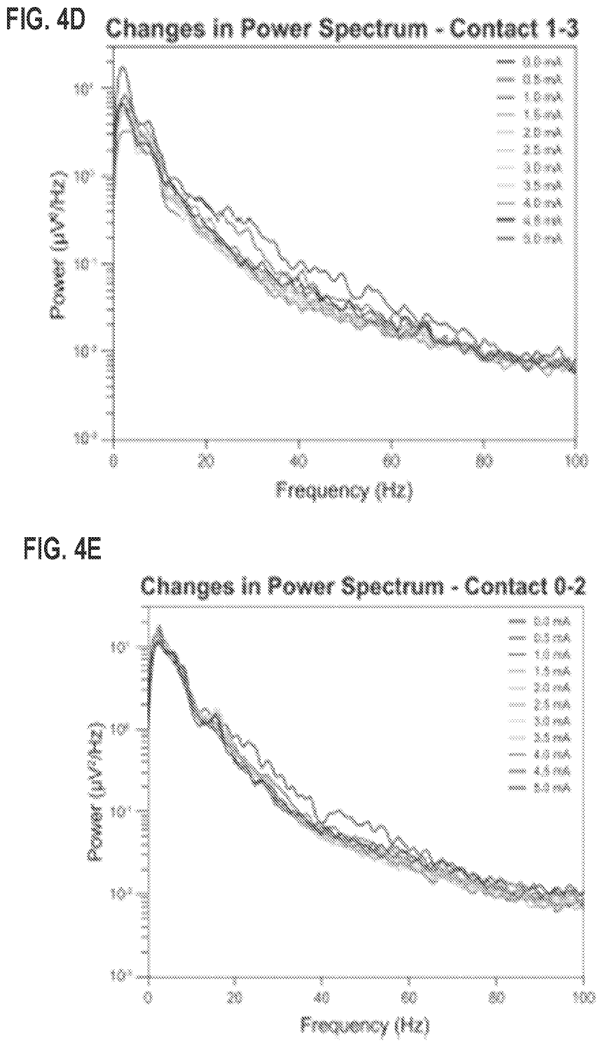

Before the stimulation signal 300 is used to stimulate the region of the brain, the apparatus 100 measures the neural signal 310 from the brain region without any stimulation. This provides a baseline neural signal 310 and corresponding Fourier transform 340 and spectrogram 350 that can be used to compare with the neural signal 310 and Fourier Transform 340 and spectrogram 350 when the brain region is being stimulated by the stimulation signal 300. FIG. 4A is a graph 400 that illustrates an example of a power spectrum indicating an average amplitude at each frequency using different non-adjacent contact pairs without stimulation, according to one embodiment. The horizontal axis 402 is frequency in units of Hertz (Hz). The vertical axis 404 is power in units of μV²/Hz.

In an embodiment, the neural signal 310 is measured during a time period when no stimulation is performed (i.e. the stimulation signal 300 is not employed). The resulting neural signal 310 is measured by one or more contacts 110 (e.g. a non-adjacent contact pair). FIG. 4A depicts three curves which each indicate an amplitude of each frequency in the frequency spectrum of the neural signal 310 detected by each of the three respective non-adjacent contact pairs 0-3, 1-3 and 0-2. In an example embodiment, curve 408 indicates the amplitude of each frequency in the frequency spectrum of the neural signal 310 detected by one non-adjacent contact pair 1-3. As shown in FIG. 4A, the curve 408 includes a beta peak 410 for non-adjacent contact pair 1-3 that indicates a maximum amplitude of the curve 408 at a beta frequency 405 in a beta frequency range (e.g. between about 13 Hz and about 39 Hz). In an example embodiment, the beta frequency 405 is about 23 Hz. As also shown in FIG. 4A, another curve 409 includes a beta peak 411 for non-adjacent contact pair 0-2 that indicates a maximum amplitude of the curve 409 at a beta frequency 407 in the beta frequency range. In an example embodiment, the beta frequency 407 is about 17 Hz. In an example embodiment, a third curve 413 indicates the amplitude of each frequency in the frequency spectrum of the neural signal 310 detected by one non-adjacent contact pair 0-3.

After determining the baseline neural signal 310 and spectrogram 350 with no stimulation of the brain region, one or more of the contacts are stimulated with the stimulation signal while other contacts (e.g. non-adjacent contact pairs) are used to measure the neural signal and spectrogram. A spectrogram similar to the spectrogram 350 (no stimulation) is generated for each set of contacts that are used to measure the neural signal. Additionally, a power spectrum similar to FIG. 4A (no stimulation) is generated which shows a curve for each stimulation amplitude.

Stimulation of the brain region with contact 2 (E02) and measurement of the neural signal with contacts 1-3 (E01-E03) will now be discussed. FIG. 4B is a graph that illustrates an example of a power spectrum indicating an average amplitude at each frequency using contact 2 based on the stimulation signal of FIGS. 3A and 3B at different amplitudes, according to one embodiment. The horizontal axis 402 and vertical axis 404 represent the same variables as in FIG. 4A. Curves 458 are provided in the graph which each indicate a respective power spectrum for a respective value of a stimulation parameter (e.g. amplitude 307). As shown in FIG. 4B, each of the curves 458 with their respective value of the stimulation parameter are distinguished using greyscale.

The graph of FIG. 4B also shows a window 425 that represents a frequency band 423 that is centered on the beta frequency 405 that corresponds to a maximum amplitude of the power spectrum curve 408 for contacts 1-3 (E01-E03) with no stimulation. In one embodiment, the frequency band 423 corresponds to a frequency range which encompasses a maximum difference between the curve 408 (power spectrum with no stimulation, also called the pathological signal) and one of the power spectrums (curves 458) corresponding to a non-zero stimulation amplitude. In an example embodiment, the frequency band 423 corresponds to the frequency range that encompasses a maximum difference between the curve 408 and the curve 458 corresponding to a maximum stimulation amplitude at which no side effects are observed in the subject. In this example embodiment, side effects in the subject are measured at each stimulation amplitude and the highest value of the stimulation amplitude at which no side effects are measured is utilized in determining the frequency band 423 (by selecting one of the curves 458 corresponding to this highest value of the stimulation amplitude). In an example embodiment, the frequency band 423 is based on a certain range (e.g. ±2 Hz) of the central frequency value (e.g. beta frequency 405).

Stimulation of the brain region with contact 1 (E01) and measurement of the neural signal with contacts 0-2 (E00-E02) will now be discussed. FIG. 4C is a graph that illustrates an example of a power spectrum indicating an average amplitude at each frequency using contact 1 based on the stimulation signal of FIGS. 3A and 3B at different amplitudes, according to one embodiment. The horizontal axis 402 and vertical axis 404 represent the same variables as in FIG. 4A. Curves 468 are provided in the graph which each indicate a respective power spectrum for a respective value of a stimulation parameter (e.g. amplitude 307). As shown in FIG. 4C, each of the curves 468 with their respective value of the stimulation parameter are distinguished using greyscale.

As with the graph of FIG. 4B, the graph of FIG. 4C also shows a window 425 that represents a frequency band 423 that is centered on the beta frequency 407 that corresponds to a maximum amplitude of the power spectrum curve 409 for contacts 0-2 (E00-E02) with no stimulation. In one embodiment, the frequency band 423 corresponds to a frequency range which encompasses a maximum difference between the curve 409 (power spectrum with no stimulation, also called the pathological signal) and one of the power spectrums (curves 468) corresponding to a non-zero stimulation amplitude. In an example embodiment, the frequency band 423 corresponds to the frequency range that encompasses a maximum difference between the curve 409 and the curve 468 corresponding to a maximum stimulation amplitude at which no side effects are observed in the subject.

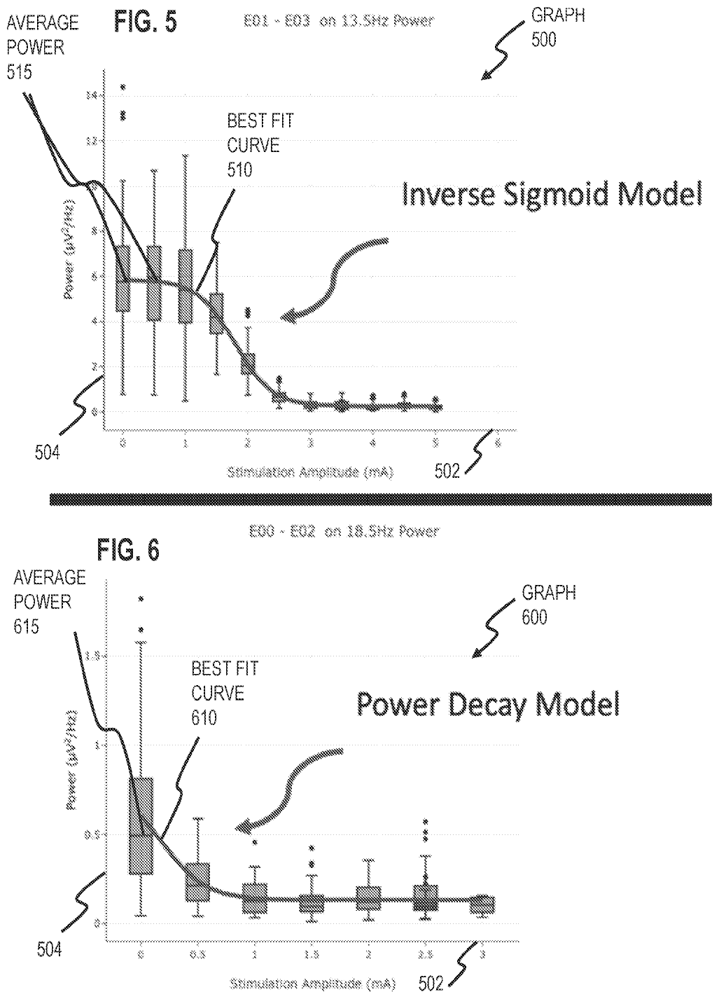

After acquiring the power spectrums at each stimulation amplitude (FIGS. 4B and 4C) and the frequency band 423, a value of a parameter of the power spectrum is then computed over the frequency band 423. In one embodiment, the value of the parameter is a value of an average of the power spectrum for each stimulation amplitude, over the frequency band 423. FIG. 5 is a graph 500 that illustrates a best fit curve of an average amplitude of the power spectrum of FIG. 4B over a frequency band for each stimulation amplitude, according to one embodiment. The horizontal axis 502 is stimulation amplitude in mA. The vertical axis 504 is power in units of μV²/Hz. As shown in FIG. 5, an average power 515 is calculated for each stimulation amplitude. In an embodiment, the average power 515 is an average amplitude of the respective curve 458 of FIG. 4B corresponding to the stimulation amplitude over the frequency band 423. In another embodiment, as shown in FIG. 5, a range of the values of the respective curve 458 of FIG. 4B corresponding to the stimulation amplitude over the frequency band 423 is indicated (e.g. by the length of the vertical line at each stimulation amplitude).

FIG. 6 is a graph 600 that illustrates an example of a best fit curve of an average amplitude of the power spectrum of FIG. 4C over a frequency band for each stimulation amplitude, according to one embodiment. The horizontal axis 502 is stimulation amplitude in mA. The vertical axis 504 is power in units of μV²/Hz. As shown in FIG. 6, an average power 615 is calculated for each stimulation amplitude. In an embodiment, the average power 615 is an average amplitude of the respective curve 468 of FIG. 4C corresponding to the stimulation amplitude over the frequency band 423. In another embodiment, as shown in FIG. 6, a range of the values of the respective curve 468 of FIG. 4C corresponding to the stimulation amplitude over the frequency band 423 is indicated (e.g. by the length of the vertical line at each stimulation amplitude).

After obtaining the average power 515, 615 (FIGS. 5, 6) data discussed above for each stimulation amplitude, a best fit curve is generated for the average power 515, 615 data for each stimulation amplitude. In one embodiment, the best fit curve is generated according to any model appreciated by one of ordinary skill in the art. In one embodiment, the best curve is generated according to the Inverse Sigmoid model based on the equation:

$$inverseSigmoidFit = \frac{L}{1 + {e}^{- k ( x - {x}_{0} )}} + {L}_{0} \qquad ( 1 )$$

where x is the value of the stimulation parameter and L, k, x₀ and L₀ are fixed values. In an embodiment, L is the coefficient of fitting and is related to how much the brain signal changed throughout the recording; Lo is the constant shift that defines the lowest neural signal in the fitted model; K is the constant of changes that defines the slope of transition from high pathological signal to low pathological signal; and X₀ is the constant shift that defines the stimulation amplitude at which the slope of transition reaches 50% (of a maximum change in the stimulation amplitude or half may between a minimum stimulation amplitude and a maximum stimulation amplitude). FIG. 5 depicts an example of a best fit curve 510 generated based on the Inverse Sigmoid model of equation 1. Although the average power 515 data was fit to the stimulation parameter data using the Inverse Sigmoid model, this data can be fit to the stimulation parameter using any best fit curve known to one of ordinary skill in the art.

In one embodiment, the best curve is generated according to the Power Decay model based on the equation:

$$log PowerFit = L * {e}^{- k ( x - {x}_{0} )} + {L}_{0} \qquad ( 2 )$$

Where x is the value of the stimulation parameter and L, k, x₀ and L₀ are fixed values that are defined above with respect to equation 1. FIG. 6 depicts an example of a best fit curve 610 generated based on the Power Decay model of equation 2. Although the average power 615 data was fit to the stimulation parameter data using the Power Decay model, this data can be fit to the stimulation parameter using any best fit curve known to one of ordinary skill in the art. In some embodiments, each of the average power 515 data or average power 615 data can be fit to the stimulation parameter data using multiple best fit models (e.g. Power Decay model and Inverse Sigmoid model). In these embodiments, a metric is determined for each best fit model (e.g. a degree to which the best fit curve for that model fits the data, such as in a percentage value) and these metrics are compared between the multiple best fit models to select one of the best fit models to generate the curve to approximate the average power data.

In an embodiment, a different model can be used to generate a best curve other than the Lower Power function and Inverse Sigmoid function. In one embodiment, this third model is a 4th power polynomial fit (e.g., Quartic function). In an example embodiment, this function will approximate the same shape as the Log Power fit of the Power Decay model. In some embodiments, the benefit of using the Quartic function is it is more stable than the exponential power function and in the event of fitting error, the quartic function is a fall back to ensure an approximate model can be used. In one embodiment, the best curve is generated according to the Quartic function model based on the equation:

$${a ( x - {x}_{0} )}^{4} + {b ( x - {x}_{0} )}^{3} + {c ( x - {x}_{0} )}^{2} + d ( x - {x}_{0} ) + e \qquad ( 3 )$$

Where x is the value of the stimulation parameter and x₀, a, b, c, d, e are fixed values.

### 2. Algorithm

A flow diagram indicating one or more steps that can be taken with the apparatus 100 is now discussed herein. FIG. 7 is a flow diagram that illustrates an example of a method 700 for determining one or more stimulation parameters of DBS. Although the flow diagram of FIG. 7 is each depicted as integral steps in a particular order for purposes of illustration, in other embodiments one or more steps, or portions thereof, are performed in a different order, or overlapping in time, in series or in parallel, or are deleted, or one or more other steps are added, or the method is changed in some combination of ways.

In step 702, first data is received by the controller 104 that indicates a value of LFP over a first time period from the contacts 110 positioned adjacent the brain region. In an embodiment, in step 702 the first data received indicates the value of the LFP over the first time period when the brain region 120, 122 is not stimulated by the contacts 110. In an example embodiment, in step 702 the neural signal 310 is measured over the first time period when the brain region 120, 122 is not stimulated by the contacts 110. In an example embodiment, step 702 is performed during the same time that the contacts 110 are surgically implanted and/or after this surgery but before the patient leaves the medical facility where the surgery was performed.

In step 704, the controller 104 receives (or determines) second data indicating a value of a parameter of a frequency spectrum of the data received in step 702. In one embodiment, in step 704 Fourier transforms 340 are generated for successive time windows 325 along the neural signal 310 (FIG. 3C). These Fourier transforms 340 are then combined into the spectrogram 350 that indicates the amplitude of the frequency spectrum of the neural signal 310 from step 702. Additionally, in one embodiment, step 704 includes generating the power spectrum (FIG. 4A) that indicates an average power of each frequency in the neural signal 310 received in step 702. In an example embodiment, in step 704 the power spectrum is generated for each respective non-adjacent contacts (e.g. non-adjacent contact pair) being used to detect the neural signal 310 in step 702.

In step 706, the controller 104 receives third data that indicates a value of the LFP over each of a plurality of time periods from one or more first contacts where the brain region is being stimulated by one or more second contacts over each respective time period based on a respective stimulation parameter value. In one embodiment, in step 706 the value of the LFP over the plurality of time periods 306 of the stimulation signal 300 is measured by some of the contacts 110 as other contacts 110 stimulate the brain region 120, 122 over the plurality of time periods 306. In an example embodiment, in step 706 the neural signal 310 is measured by some of the contacts 110 as the brain region is being stimulated by other contacts 110.

In step 708, the controller 104 receives fourth data that indicates a value of a plurality of second frequency spectrums of the third data received in step 706 for each stimulation parameter value. As shown in FIGS. 3A and 3B, for each time period 306, the amplitude 307 of the stimulation signal 300 is varied and consequently the frequency spectrum of the neural signal 310 is varied. In one embodiment, in step 708 a respective spectrogram (such as spectrogram 350 of FIG. 3E) and power spectrum (such as FIG. 4B or 4C) is determined for each time period 306 and consequently for each stimulation amplitude 307 value.

In step 710, a frequency band is determined that encompasses a characteristic of a difference between a value of a parameter of the first frequency spectrum (from step 704) and a value of a parameter of one of the second frequency spectrums (from step 708). In one embodiment, the frequency band is defined by a central frequency value (e.g. beta frequency 405) that corresponds to a maximum value of the first frequency spectrum and a certain range outside that central frequency value (e.g. ±2 Hz). In another embodiment, the value of the parameter of the first frequency spectrum is the value of a peak (e.g. beta peak 410 at the beta frequency 405 of FIG. 4A) and the value of the parameter of one of the second frequency spectrums is a value of the second frequency spectrum amplitude at the same frequency that corresponds to the peak value in the first frequency spectrum (e.g. the value of one of the curves 458 that corresponds to the beta frequency 405). In some embodiments, during step 706 data is collected with respect to a degree of symptoms (side effects) of the subject in response to the varying of the stimulation amplitude. In these embodiments, the largest stimulation amplitude at which no symptoms were observed is utilized in step 710 when selecting the second frequency spectrum to compute the difference. However, in other embodiments, other second frequency spectrums (e.g. other non-zero stimulation amplitudes) can be used to compute the difference.

In step 712, a value of a parameter of each second frequency spectrum over the frequency band (from step 710) is determined for each stimulation parameter value. In an embodiment, in step 712 an average amplitude and/or a range of amplitudes of each second frequency spectrum over the frequency band 423 is determined for each stimulation amplitude value. In one embodiment, in step 712 the average power 515 values are determined for each stimulation amplitude value (e.g. for contacts 1-3 or E01-E03). In another embodiment, in step 712 the average power 615 values are determined for each stimulation amplitude value (e.g. for contacts 0-2 or E00-E02).

In step 714, a curve is determined that indicates a best fit between the values of the parameter of each second frequency spectrum (from step 712) and each stimulation parameter value. In one embodiment, in step 714 the best fit curve 510 is generated using the Inverse Sigmoid model of equation 1. In another embodiment, in step 714 the best fit curve 610 is generated using the Power Decay model of equation 2. In another embodiment, in step 714 the best fit curve is generated using the Quartic function model of equation 3. In some embodiments, in step 714 multiple best fit curves are generated for the data from step 712 using multiple models. In these embodiments, a metric is determined that indicates a degree of fit (e.g. as a percentage value) between the curves and the parameter values of each second frequency spectrum. In these embodiments, the curve which has the highest metric value is utilized in step 716.

In step 716, a value of a feature of the curve from step 714 is determined and is used to determine a value of one or more optimal stimulation parameters. In an embodiment, the feature of the curve is a maximum change in amplitude of the curve (e.g. across the range of stimulation amplitudes). In an example embodiment, this maximum change of the curve 510 in FIG. 5 is about 6 μV²/Hz whereas this maximum change of the curve 610 in FIG. 6 is about 0.5 μV²/Hz. In another embodiment, the feature of the curve is the value of the spectral power before the patient experiences stimulation-induced side effects. In this embodiment, in step 716 as the stimulation amplitude is varied one or more symptoms (e.g. side effects) of the subject are measured and a largest stimulation amplitude before a subject experiences side effects is recorded. Thus, in this embodiment, this feature of the curve determined in step 716 is a value of the curve (e.g. power value along the vertical axis 504) that corresponds to this threshold stimulation amplitude (e.g. just before the subject experiences side-effects). In yet another embodiment, the feature of the curve determined in step 716 is the stimulation amplitude (e.g. along axis 502) where the value of the curve reduces to a low threshold percentage (e.g. 5%) of its maximum value (e.g. at 0 mA stimulation amplitude). In yet another embodiment, a feature of the curve determined in step 716 is an available therapeutic window (e.g. range of stimulation amplitude along the axis 502) at which the subject can be effectively stimulated to achieve therapeutic results. In this example embodiment, the therapeutic window has a minimum stimulation amplitude (e.g. corresponding to the stimulation amplitude where the value of the curve has fallen to a threshold percentage, e.g. 5%, of the maximum value) and a maximum stimulation amplitude (e.g. just below the threshold stimulation amplitude at which side effects are observed).

In step 716, the value of the optimal stimulation parameter (e.g. optimal stimulation amplitude) is determined based on the determined values of the feature of the curve. Although the above embodiments discuss determining a value of the stimulation amplitude 307, in other embodiments any of the other stimulation parameter values (e.g. pulse width 303, recharge phase 305 duration, frequency 309) could be determined in this step where the other steps of the method 700 are performed where this other stimulation parameter is varied between the time periods 306 of the stimulation signal 300.

Although steps 714 and 716 discuss generating the best fit curve and then determining one or more features of the curve, in other embodiments the best fit curve and features of the best fit curve need not be generated and instead the value of optimal stimulation parameters in step 716 is just based on the data obtained in step 712 (e.g. manually observing the values of the data obtained in step 712 to visually approximate the stimulation amplitude at which the power is reduced to a minimum value or near a minimum value).

Although step 716 discusses determining one or more optimal stimulation parameter values, in other embodiments step 716 is used to determine an optimal stimulation contact 110 to utilize in stimulating the brain region. In one embodiment, FIGS. 5 and 6 show best fit curves 510, 610 using different stimulation contacts (e.g. contact 2 or E02 in FIG. 5; contact 1 or E01 in FIG. 6). In these embodiments, in step 716 an optimal stimulation contact is selected (between contact 1/E01 and 2/E02) based on the determined features of the curves 510, 610 in step 716. For example, in step 716 contact 2 or E02 may be selected as the optimal stimulation contact, since the best fit curve 510 is reduced by a greater power value (e.g. about 6 μV²/Hz) than best fit curve 610 (e.g. about 0.5 μV²/Hz). However, in other embodiments, in step 716 contact 1 or E01 is selected since the curve 610 indicates that the power is reduced to 5% of the maximum value of the curve 610 at a smaller stimulation amplitude (e.g. about 1 mA) as compared with curve 510 (e.g. about 3 mA) and thus involves a smaller risk of inducing side effects.

In one embodiment, in step 716 one or more optimal stimulation parameters are determined including an optimal stimulation contact, an optimal stimulation frequency 309, an optimal amplitude 307 and an optimal pulse width 303. In an example embodiment, based on the best fit curve 510 of FIG. 5, these parameters include selecting the contact 2/E02, a frequency 309 of about 135 Hz, an amplitude 307 of about 2.65 mA and a pulse width of about 90 μs. In an example embodiment, based on the best fit curve 610 of FIG. 6, these parameters include selecting the contact 1/E01, a frequency 309 of about 125 Hz, an amplitude 307 of about 0.83 mA and a pulse width of about 60 μs. In these embodiments, the optimal stimulation amplitude 307 based on the best fit curve 610 is based on the curve 610 reaching a minimal power (e.g. 5% of the maximum value at 0 mA) at a much smaller stimulation amplitude than the best fit curve 510.

In one embodiment, in step 716 the optimal stimulation contact is determined by building a support vector machine (SVM) classifier. In an embodiment, the SVM classifier is determined based on features obtained from existing subjects and labels created by DBS programming experts regarding which stimulation contact provides a beneficial outcome. In an example embodiment, the SVM classifier is obtained by performing the method 700 with multiple subjects. During an initial reading of each subject, steps 702 through 706 are performed in order to measure the neural signals 310 with no stimulation amplitude and at increasing stimulation amplitude values. In these embodiments, steps 708 through 716 are then performed for each recording to extract the model features (e.g. determine the best fit curve and values of the best fit curve features). An expert then provides a rating for each recording based on whether it is therapeutically beneficial (“good outcome”) or not (“bad outcome”). Then a kernel SVM (e.g. radial basis function SVM) is built based on these extracted model features and expert ratings for each reading (e.g. each iteration of the method 700). The resulting SVM classifier is then applied to future readings (e.g. future iterations of the method 700) and a probability of a therapeutically beneficial outcome is computed. In these embodiments, the probability of the therapeutically beneficial outcome is determined by comparing the extracted model features of the new reading with the previously-known extracted model features for the subject corresponding to a “good outcome” rating. For example, if a new reading extracts model features which are 95% similar to a previously recorded reading having a “good outcome” for the subject, then this new reading may receive a 95% probability of having a good therapeutic outcome.

### 3. Example Embodiments

A number of studies have shown that STN beta power (13-30 Hz) is correlated with Parkinson's disease (PD) symptom severity, particularly rigidity and bradykinesia [1, 2], and that the pathological beta signal will be attenuated by effective therapeutic STN stimulation [3]. Recent advancements in the DBS technology have provided broader access to neural activity near the electrode and access during actual stimulation [4]. This advance has the potential to facilitate translation of existing research findings into the clinical environment and to enable the development of more objective approaches for DBS programming. Using a chronic sensing-enabled neurostimulator recently approved for commercial use (Medtronic Percept®), robust changes were observed in the STN beta power during stepwise monopolar stimulation which could be used as a method to select optimal therapeutic settings [5]. Although GPi DBS is increasingly used for the treatment of PD, with a number of intraoperative pallidal physiology studies and chronic recordings shown potential correlates of symptoms, chronic sensing GPi physiology remains largely unknown and its potential to assist in determining optimal therapeutic programming settings remains to be demonstrated.

In an example embodiment, a single PD participant receiving unilateral GPi DBS treatment was implanted with a novel sensing-enabled neurostimulator. In this example embodiment, in step 702 the neural signals were recorded in the GPi region during DBS treatment and in the method 700 the relationship between the changes in neural signals and symptom improvement were analyzed. It was determined whether pallidal stimulation would reduce the beta power and correlate with symptom improvement, in which case the embodiments disclosed herein might be useful as an objective measure to guide future DBS programming.

In an example embodiment, the study participant is a 57-year old male with a 10 years history of PD. The study participant was recruited and consented according to the Declaration of Helsinki. The participant was diagnosed as the akinetic and rigid subtype of PD with minimal tremor symptoms. The participant was on 1100 mg L-Dopa equivalent daily dose of PD medication prior to receiving the DBS therapy. The Unified Parkinson's Disease Rating Scale (UPDRS) Part III motor score at baseline was 34 off medication and 21 on medication. The participant was implanted with unilateral GPi DBS of left hemisphere.

In an example embodiment, a DBS electrode 110 (e.g., Medtronic Model 3387® was implanted in the GPi. The surgical planning was performed using a modified digital Schaltenbrand-Bailey deformable atlas overlay over an MRI FGATIR sequence for targeting and microelectrode recording was performed along the planned trajectory to verify the presence of GPi neurons along the trajectory. A month following the DBS electrode implantation, the impulse generator 102 (e.g., Medtronic Percept®), a chronic sensing-enabled neurostimulator, was implanted into the right sub-clavicular region. This DBS device is a commercially available non-rechargeable neurostimulator delivering constant current and was enabled for LFPs sensing. The final position of the DBS electrode was verified using postoperative non-contrast CT head imaging which was fused with the pre-operative MRI FGATIR using a 3D affine transformation. The same modified digital Schaltenbrand-Bailey atlas used for DBS implantation surgery was used to verify the electrode position relative to the target region.

In an example embodiment, electrophysiological recordings were collected during the initial monopolar review visit 1 week following neurostimulator implantation. The electrophysiological recordings were collected with a sampling rate of 250 Hz as limited by the neurostimulator. In an example embodiment, in steps 702 and 704, a 2-min baseline LFP recording off stimulation was performed to determine the baseline characteristics of the three non-adjacent bipolar recording contact pairs (0-2, 1-3, and 0-3). Then, in step 706, the clinical programmer examined the effect of stimulation delivered at each contact sequentially from the most ventral to the most dorsal contact by slowly increasing the stimulation amplitude while pulse width and frequency were kept constant at 90 mS and 130 Hz until the participant reported persistent stimulation induced side effects. During stimulation testing using contact 1 and 2, in step 706 the LFPs were recorded continuously from contact 0-2 and 1-3, respectively. The stimulation amplitude was increased by 0.5 mA increments starting at 0 mA up to the side effects threshold, with a minimum of a 20 s waiting period between each amplitude change. In addition to the LFP, in step 708 spectral power at the pre-defined frequency was simultaneously recorded, with a 1.26 ms sensing blanking and was averaged over 3000 ms. In step 710, the power band was defined as the maximum beta peak at rest (17.57±2 Hz for contact 0-2 and 23.44±2 Hz for contact 1-3) by visual inspection on the Medtronic programming tablet. The defined spectral band power was collected by Percept PC at a rate of 2 Hz (500 ms per data point) with 3000 ms averaging window. After the waiting period, the clinician performed an assessment of contralateral upper limb rigidity and bradykinesia using the UPDRS item 22 and 23, respectively, to evaluate the therapeutic benefit of each stimulation amplitude.

In an example embodiment, the LFPs collected in steps 702 and 706 were exported to a standard JSON format file that was parsed and imported to Python 3. The raw time domain LFP recordings were filtered between 1 and 100 Hz to remove drift and stimulation artifacts, then transformed to time frequency plots in steps 704 and 708 using standard spectrogram with 1-s Hamming window and 0.5-s overlap. To quantify the stimulation-induced power changes, 15 s of data free of artifact, excluding 2 s of data immediately before and after stimulation changes were used to avoid stimulation transition artifacts; these were selected at each stimulation amplitude and the power spectral density was computed using Welch's periodogram method with 1-s hamming window and 0.5-s overlap. The beta power which was simultaneously streamed was also averaged at each stimulation amplitude and was correlated with the symptom severity score from UPDRS motor assessment by applying a Spearman's correlation.

In an example embodiment, FIG. 4A shows the power spectral density computed from the three sensing-enabled stimulation contact pairs (0-2, 1-3, and 0-3). A large peak 410 in the beta band (13-30 Hz) was observed in bipolar sensing contact pairs 1-3, with the maximum power occurring at 23.44 Hz. Contact pairs 0-2 and 0-3 had minimal activity in the beta band and were instead characterized by stronger low frequency oscillations (1-10 Hz). These results suggested that contact 1 was likely localized closer to the beta source. FIGS. 2A through 2D show the final electrode positions relative to the GPi. The most ventral contact (contact 0) was inferior to the GPi border, contacts 1 and 2 were within the GPi and the most dorsal contact (contact 3) was located on the border of GPi and GPe.

In an example embodiment, FIGS. 3F and 3G show the time-frequency plots of LFPs recorded from contact pair 1-3 while delivering stimulation from contact 2. The stimulation was slowly increased by a 0.5 mA increment from 0 mA to 5 mA, and hand tingling and mouth pulling was induced by the procedure at higher thresholds. The incremental stimulation amplitude is shown in FIG. 3G. Note that the stimulation changes were associated with transient artifacts. To quantify the effect of stimulation, the PSDs were computed at each stimulation amplitude, excluding a 2 s period at the stimulation transitions, and this is shown on FIG. 4D. A reduction in beta and low gamma power with increased GPi stimulation was observed and shown in FIGS. 3F, 3G and 4D. Interestingly, a slight rebound in beta power was observed when stimulation amplitude was closer to the side-effect threshold.

In an example embodiment, FIGS. 3H and 3I show the time-frequency plots of LFPs recorded from contact pair 0-2 while delivering stimulation from contact 1. Similar stepwise increment was performed at contact 1 and the stimulation PSDs were shown in FIG. 4E. A narrow beta peak was observed at 17 Hz, but this peak was not correlated with symptom improvement. A reduction in broader high beta power was also found when stimulating from contact 1 and recording LFPs from contact 0-2 (FIG. 4E) but not as significant as contact 2.

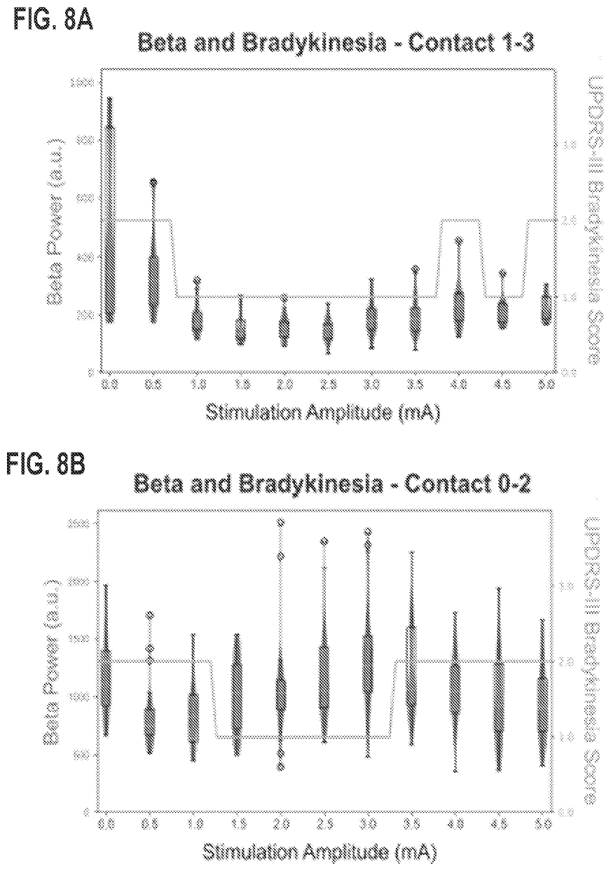

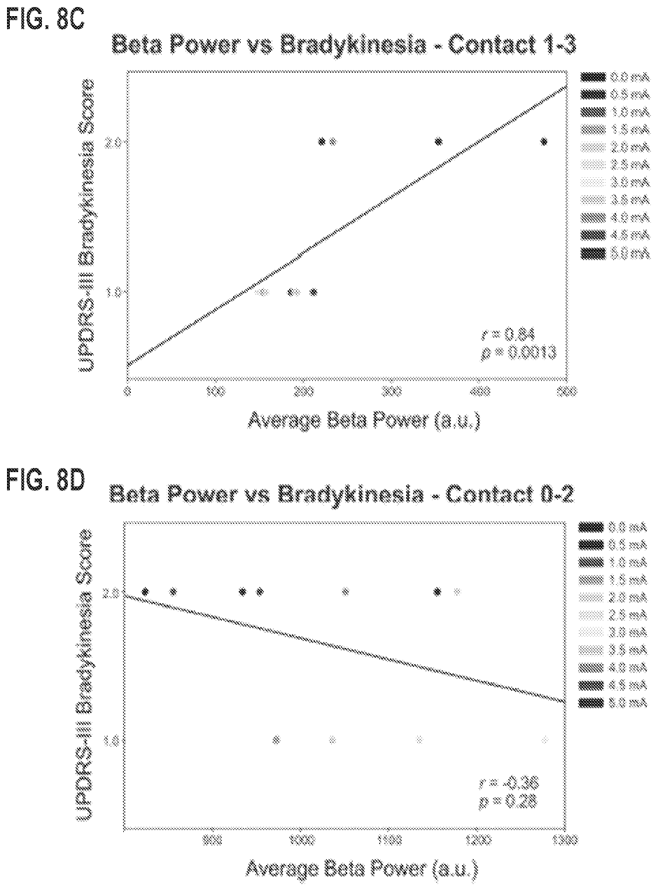

In an example embodiment, of the two primary symptom measurements, only bradykinesia shows improvement with stimulation while rigidity remain at a score of 1 throughout the full therapy range; therefore, rigidity was not reported in the correlation analysis. FIGS. 8A and 8B show both the distribution and the median of the average beta power (17.57±2 Hz for contact 0-2 and 23.44±2 Hz for contact 1-3) at each stimulation amplitude and this is displayed as a box violin plot for LFP recorded from contacts 0-2 while stimulating on contact 1 and contacts 1-3 while stimulating on contact 2. Contact 2 best controlled the participant's symptoms. Increased stimulation amplitude resulted in a reduction in beta power, with a considerable change at 1 mA at contact 2. Interestingly, the participant experienced a worsening of bradykinesia with a stimulation amplitude above 4.0 mA and the worsening was associated with increased beta power. The bradykinesia scores across stimulation amplitudes (FIG. 8A) were significantly correlated with the average beta power values (FIG. 8C) and analysis with a Spearman's correlation coefficient revealed 0.84 (p=0.0013). Contact 1 LFP does not show correlation with symptom improvement (FIG. 8D, p=0.28) and contact 1 therapy has a narrower therapeutic window than contact 2 (FIG. 8B).

In an example embodiment, in this study a novel sensing-enabled DBS device was applied to an individual with PD implanted with GPi DBS. In step 706, LFPs were recorded while delivering stimulation, at contact 1 and 2 sequentially, and by slowly increasing stimulation amplitude until side effects were encountered. The bradykinesia severity and rigidity were recorded at each stimulation amplitude and was correlated with the LFP feature.

In an example embodiment, real-time neuronal recordings during threshold testing revealed a beta desynchronization when electrical stimulation was delivered to the target region. The beta desynchronization was stronger for the sensing contact pair 1-3 than for contact pair 0-2. This matched the baseline characteristics which also revealed a stronger beta peak when sensing at contact pair 1-3. This result matched with the imaging which revealed that the electrode contacts were located within the GPi. The beta power was strongly correlated with bradykinesia improvement when measurements occurred in the acute clinic setting. However, rigidity score was not changed by the stimulation in either therapy contact pairs. In this example embodiment, these results suggest that GPi beta power correlates with bradykinesia severity and might be used as an objective marker for selecting the optimal stimulation amplitude for treatment of bradykinesia.

In an example embodiment, FIGS. 3F and 3G indicate time-frequency analysis during stimulation threshold testing and their corresponding stimulation amplitude is displayed. Note the stimulation amplitudes are up to but not exceeding the side-effect threshold for stimulation contact 2. In an example embodiment, FIG. 4D shows different greyscale for different curves that denote the absolute power at each time point, with dark grey indicating strongest power and light grey the lowest. Sensing contact 1-3 revealed a reduction of power in the beta band in spectrogram when the stimulation amplitude was increased.

In an example embodiment, FIGS. 3H and 3I indicate increased stimulation amplitude was associated with a decrease in beta band power. The same time-frequency analysis was repeated for contact 1 as comparison. In an example embodiment, FIG. 4E shows that increased stimulation amplitude was associated with a smaller decrease in beta band power.

In an example embodiment, FIGS. 8A and 8C show a relationship between beta power (recorded from contact 1-3 when stimulating at contact 2) and bradykinesia score (UPDRS Part 3 subitem 23). In another example embodiment, FIG. 8A shows that the beta peak power (23.44 Hz) for contact 1-3 was averaged over each stimulation amplitude and shown in a box-violin plot with whisker at 150% of the interquartile distance and outliers were marked as circles. Each datapoint indicates a 500 ms power value collected by Medtronic Percept neurostimulator at the specified frequency band. The bradykinesia scores were plotted in the same scale for comparison. In an example embodiment, FIG. 8C shows the correlation between average beta power and bradykinesia score at each stimulation amplitude for contact 1-3. The beta power was statistically significantly correlated with bradykinesia scores, with increased beta power correlated with worse bradykinesia (p=0.0013).

In an example embodiment, FIG. 8B shows that the beta peak power (17.57 Hz) for contact 0-2 was averaged over each stimulation amplitude and shown in a box-violin plot with whisker at 150% of the interquartile distance and outliers were marked as circle. In an example embodiment, FIG. 8D shows the correlation between average beta power and bradykinesia score at each stimulation amplitude for contact 0-2.

In an example embodiment, this procedure could be practical and useful for clinic-based settings. Interestingly, the beta power rebounded when the stimulation amplitude was increased above 3 mA, and this rebound was associated with an increase in bradykinesia severity. The rebound of the beta power can provide useful information regarding the therapeutic window for DBS programming in addition to finding to therapy level that provide maximum benefits.

### 4. Hardware Overview

FIG. 9 is a block diagram that illustrates a computer system 900 upon which an embodiment of the invention may be implemented. Computer system 900 includes a communication mechanism such as a bus 910 for passing information between other internal and external components of the computer system 900. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 900, or a portion thereof, constitutes a means for performing one or more steps of one or more methods described herein.

A sequence of binary digits constitutes digital data that is used to represent a number or code for a character. A bus 910 includes many parallel conductors of information so that information is transferred quickly among devices coupled to the bus 910. One or more processors 902 for processing information are coupled with the bus 910. A processor 902 performs a set of operations on information. The set of operations include bringing information in from the bus 910 and placing information on the bus 910. The set of operations also typically include comparing two or more units of information, shifting positions of units of information, and combining two or more units of information, such as by addition or multiplication. A sequence of operations to be executed by the processor 902 constitutes computer instructions.

Computer system 900 also includes a memory 904 coupled to bus 910. The memory 904, such as a random access memory (RAM) or other dynamic storage device, stores information including computer instructions. Dynamic memory allows information stored therein to be changed by the computer system 900. RAM allows a unit of information stored at a location called a memory address to be stored and retrieved independently of information at neighboring addresses. The memory 904 is also used by the processor 902 to store temporary values during execution of computer instructions. The computer system 900 also includes a read only memory (ROM) 906 or other static storage device coupled to the bus 910 for storing static information, including instructions, that is not changed by the computer system 900. Also coupled to bus 910 is a non-volatile (persistent) storage device 908, such as a magnetic disk or optical disk, for storing information, including instructions, that persists even when the computer system 900 is turned off or otherwise loses power.

Information, including instructions, is provided to the bus 910 for use by the processor from an external input device 912, such as a keyboard containing alphanumeric keys operated by a human user, or a sensor. A sensor detects conditions in its vicinity and transforms those detections into signals compatible with the signals used to represent information in computer system 900. Other external devices coupled to bus 910, used primarily for interacting with humans, include a display device 914, such as a cathode ray tube (CRT) or a liquid crystal display (LCD), for presenting images, and a pointing device 916, such as a mouse or a trackball or cursor direction keys, for controlling a position of a small cursor image presented on the display 914 and issuing commands associated with graphical elements presented on the display 914.

In the illustrated embodiment, special purpose hardware, such as an application specific integrated circuit (IC) 920, is coupled to bus 910. The special purpose hardware is configured to perform operations not performed by processor 902 quickly enough for special purposes. Examples of application specific ICs include graphics accelerator cards for generating images for display 914, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 900 also includes one or more instances of a communications interface 970 coupled to bus 910. Communication interface 970 provides a two-way communication coupling to a variety of external devices that operate with their own processors, such as printers, scanners and external disks. In general the coupling is with a network link 978 that is connected to a local network 980 to which a variety of external devices with their own processors are connected. For example, communication interface 970 may be a parallel port or a serial port or a universal serial bus (USB) port on a personal computer. In some embodiments, communications interface 970 is an integrated services digital network (ISDN) card or a digital subscriber line (DSL) card or a telephone modem that provides an information communication connection to a corresponding type of telephone line. In some embodiments, a communication interface 970 is a cable modem that converts signals on bus 910 into signals for a communication connection over a coaxial cable or into optical signals for a communication connection over a fiber optic cable. As another example, communications interface 970 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 970 sends and receives electrical, acoustic or electromagnetic signals, including infrared and optical signals, that carry information streams, such as digital data.

The term computer-readable medium is used herein to refer to any medium that participates in providing information to processor 902, including instructions for execution. Such a medium may take many forms, including, but not limited to, non-volatile media, volatile media and transmission media. Non-volatile media include, for example, optical or magnetic disks, such as storage device 908. Volatile media include, for example, dynamic memory 904. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 902, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 902, except for carrier waves and other signals.

Logic encoded in one or more tangible media includes one or both of processor instructions on a computer-readable storage media and special purpose hardware, such as ASIC *920.

Network link 978 typically provides information communication through one or more networks to other devices that use or process the information. For example, network link 978 may provide a connection through local network 980 to a host computer 982 or to equipment 984 operated by an Internet Service Provider (ISP). ISP equipment 984 in turn provides data communication services through the public, world-wide packet-switching communication network of networks now commonly referred to as the Internet 990. A computer called a server 992 connected to the Internet provides a service in response to information received over the Internet. For example, server 992 provides information representing video data for presentation at display 914.

The invention is related to the use of computer system 900 for implementing the techniques described herein. According to one embodiment of the invention, those techniques are performed by computer system 900 in response to processor 902 executing one or more sequences of one or more instructions contained in memory 904. Such instructions, also called software and program code, may be read into memory 904 from another computer-readable medium such as storage device 908. Execution of the sequences of instructions contained in memory 904 causes processor 902 to perform the method steps described herein. In alternative embodiments, hardware, such as application specific integrated circuit 920, may be used in place of or in combination with software to implement the invention. Thus, embodiments of the invention are not limited to any specific combination of hardware and software.

The signals transmitted over network link 978 and other networks through communications interface 970, carry information to and from computer system 900. Computer system 900 can send and receive information, including program code, through the networks 980, 990 among others, through network link 978 and communications interface 970. In an example using the Internet 990, a server 992 transmits program code for a particular application, requested by a message sent from computer 900, through Internet 990, ISP equipment 984, local network 980 and communications interface 970. The received code may be executed by processor 902 as it is received, or may be stored in storage device 908 or other non-volatile storage for later execution, or both. In this manner, computer system 900 may obtain application program code in the form of a signal on a carrier wave.

Various forms of computer readable media may be involved in carrying one or more sequence of instructions or data or both to processor 902 for execution. For example, instructions and data may initially be carried on a magnetic disk of a remote computer such as host 982. The remote computer loads the instructions and data into its dynamic memory and sends the instructions and data over a telephone line using a modem. A modem local to the computer system 900 receives the instructions and data on a telephone line and uses an infra-red transmitter to convert the instructions and data to a signal on an infra-red a carrier wave serving as the network link 978. An infrared detector serving as communications interface 970 receives the instructions and data carried in the infrared signal and places information representing the instructions and data onto bus 910. Bus 910 carries the information to memory 904 from which processor 902 retrieves and executes the instructions using some of the data sent with the instructions. The instructions and data received in memory 904 may optionally be stored on storage device 908, either before or after execution by the processor 902.

FIG. 10 illustrates a chip set 1000 upon which an embodiment of the invention may be implemented. Chip set 1000 is programmed to perform one or more steps of a method described herein and includes, for instance, the processor and memory components described with respect to FIG. *9 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 1000, or a portion thereof, constitutes a means for performing one or more steps of a method described herein.

In one embodiment, the chip set 1000 includes a communication mechanism such as a bus 1001 for passing information among the components of the chip set 1000. A processor 1003 has connectivity to the bus 1001 to execute instructions and process information stored in, for example, a memory 1005. The processor 1003 may include one or more processing cores with each core configured to perform independently. A multi-core processor enables multiprocessing within a single physical package. Examples of a multi-core processor include two, four, eight, or greater numbers of processing cores. Alternatively or in addition, the processor 1003 may include one or more microprocessors configured in tandem via the bus 1001 to enable independent execution of instructions, pipelining, and multithreading. The processor 1003 may also be accompanied with one or more specialized components to perform certain processing functions and tasks such as one or more digital signal processors (DSP) 1007, or one or more application-specific integrated circuits (ASIC) 1009. A DSP 1007 typically is configured to process real-world signals (e.g., sound) in real time independently of the processor 1003. Similarly, an ASIC 1009 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 1003 and accompanying components have connectivity to the memory 1005 via the bus 1001. The memory 1005 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 1005 also stores the data associated with or generated by the execution of one or more steps of the methods described herein.

FIG. 11 is a diagram of exemplary components of a mobile terminal 1100 (e.g., cell phone handset) for communications, which is capable of operating in the system of FIG. 2C, according to one embodiment. In some embodiments, mobile terminal 1101, or a portion thereof, constitutes a means for performing one or more steps described herein. Generally, a radio receiver is often defined in terms of front-end and back-end characteristics. The front-end of the receiver encompasses all of the Radio Frequency (RF) circuitry whereas the back-end encompasses all of the base-band processing circuitry. As used in this application, the term “circuitry” refers to both: (1) hardware-only implementations (such as implementations in only analog and/or digital circuitry), and (2) to combinations of circuitry and software (and/or firmware) (such as, if applicable to the particular context, to a combination of processor(s), including digital signal processor(s), software, and memory(ies) that work together to cause an apparatus, such as a mobile phone or server, to perform various functions). This definition of “circuitry” applies to all uses of this term in this application, including in any claims. As a further example, as used in this application and if applicable to the particular context, the term “circuitry” would also cover an implementation of merely a processor (or multiple processors) and its (or their) accompanying software/or firmware. The term “circuitry” would also cover if applicable to the particular context, for example, a baseband integrated circuit or applications processor integrated circuit in a mobile phone or a similar integrated circuit in a cellular network device or other network devices.

Pertinent internal components of the telephone include a Main Control Unit (MCU) 1103, a Digital Signal Processor (DSP) 1105, and a receiver/transmitter unit including a microphone gain control unit and a speaker gain control unit. A main display unit 1107 provides a display to the user in support of various applications and mobile terminal functions that perform or support the steps as described herein. The display 1107 includes display circuitry configured to display at least a portion of a user interface of the mobile terminal (e.g., mobile telephone). Additionally, the display 1107 and display circuitry are configured to facilitate user control of at least some functions of the mobile terminal. An audio function circuitry 1109 includes a microphone 1111 and microphone amplifier that amplifies the speech signal output from the microphone 1111. The amplified speech signal output from the microphone 1111 is fed to a coder/decoder (CODEC) 1113.

A radio section 1115 amplifies power and converts frequency in order to communicate with a base station, which is included in a mobile communication system, via antenna 1117. The power amplifier (PA) 1119 and the transmitter/modulation circuitry are operationally responsive to the MCU 1103, with an output from the PA 1119 coupled to the duplexer 1121 or circulator or antenna switch, as known in the art. The PA 1119 also couples to a battery interface and power control unit 1120.

In use, a user of mobile terminal 1101 speaks into the microphone 1111 and his or her voice along with any detected background noise is converted into an analog voltage. The analog voltage is then converted into a digital signal through the Analog to Digital Converter (ADC) 1123. The control unit 1103 routes the digital signal into the DSP 1105 for processing therein, such as speech encoding, channel encoding, encrypting, and interleaving. In one embodiment, the processed voice signals are encoded, by units not separately shown, using a cellular transmission protocol such as enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., microwave access (WiMAX), Long Term Evolution (LTE) networks, code division multiple access (CDMA), wideband code division multiple access (WCDMA), wireless fidelity (WiFi), satellite, and the like, or any combination thereof.

The encoded signals are then routed to an equalizer 1125 for compensation of any frequency-dependent impairments that occur during transmission though the air such as phase and amplitude distortion. After equalizing the bit stream, the modulator 1127 combines the signal with a RF signal generated in the RF interface 1129. The modulator 1127 generates a sine wave by way of frequency or phase modulation. In order to prepare the signal for transmission, an up-converter 1131 combines the sine wave output from the modulator 1127 with another sine wave generated by a synthesizer 1133 to achieve the desired frequency of transmission. The signal is then sent through a PA 1119 to increase the signal to an appropriate power level. In practical systems, the PA 1119 acts as a variable gain amplifier whose gain is controlled by the DSP 1105 from information received from a network base station. The signal is then filtered within the duplexer 1121 and optionally sent to an antenna coupler 1135 to match impedances to provide maximum power transfer. Finally, the signal is transmitted via antenna 1117 to a local base station. An automatic gain control (AGC) can be supplied to control the gain of the final stages of the receiver. The signals may be forwarded from there to a remote telephone which may be another cellular telephone, any other mobile phone or a land-line connected to a Public Switched Telephone Network (PSTN), or other telephony networks.

Voice signals transmitted to the mobile terminal 1101 are received via antenna 1117 and immediately amplified by a low noise amplifier (LNA) 1137. A down-converter 1139 lowers the carrier frequency while the demodulator 1141 strips away the RF leaving only a digital bit stream. The signal then goes through the equalizer 1125 and is processed by the DSP 1105. A Digital to Analog Converter (DAC) 1143 converts the signal and the resulting output is transmitted to the user through the speaker 1145, all under control of a Main Control Unit (MCU) 1103 which can be implemented as a Central Processing Unit (CPU) (not shown).

The MCU 1103 receives various signals including input signals from the keyboard 1147. The keyboard 1147 and/or the MCU 1103 in combination with other user input components (e.g., the microphone 1111) comprise a user interface circuitry for managing user input. The MCU 1103 runs a user interface software to facilitate user control of at least some functions of the mobile terminal 1101 as described herein. The MCU 1103 also delivers a display command and a switch command to the display 1107 and to the speech output switching controller, respectively. Further, the MCU 1103 exchanges information with the DSP 1105 and can access an optionally incorporated SIM card 1149 and a memory 1151. In addition, the MCU 1103 executes various control functions required of the terminal. The DSP 1105 may, depending upon the implementation, perform any of a variety of conventional digital processing functions on the voice signals. Additionally, DSP 1105 determines the background noise level of the local environment from the signals detected by microphone 1111 and sets the gain of microphone 1111 to a level selected to compensate for the natural tendency of the user of the mobile terminal 1101.

The CODEC 1113 includes the ADC 1123 and DAC 1143. The memory 1151 stores various data including call incoming tone data and is capable of storing other data including music data received via, e.g., the global Internet. The software module could reside in RAM memory, flash memory, registers, or any other form of writable storage medium known in the art. The memory device 1151 may be, but not limited to, a single memory, CD, DVD, ROM, RAM, EEPROM, optical storage, magnetic disk storage, flash memory storage, or any other non-volatile storage medium capable of storing digital data.

An optionally incorporated SIM card 1149 carries, for instance, important information, such as the cellular phone number, the carrier supplying service, subscription details, and security information. The SIM card 1149 serves primarily to identify the mobile terminal 1101 on a radio network. The card 1149 also contains a memory for storing a personal telephone number registry, text messages, and user specific mobile terminal settings.

In some embodiments, the mobile terminal 1101 includes a digital camera comprising an array of optical detectors, such as charge coupled device (CCD) array 1165. The output of the array is image data that is transferred to the MCU for further processing or storage in the memory 1151 or both. In the illustrated embodiment, the light impinges on the optical array through a lens 1163, such as a pin-hole lens or a material lens made of an optical grade glass or plastic material. In the illustrated embodiment, the mobile terminal 1101 includes a light source 1161, such as a LED to illuminate a subject for capture by the optical array, e.g., CCD 1165. The light source is powered by the battery interface and power control module 1120 and controlled by the MCU 1103 based on instructions stored or loaded into the MCU 1103.

### 5. Alternatives, Deviations and Modifications

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5X to 2X, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

### 6. REFERENCES

[1] Weinberger, M., Mahant, N., Hutchison, W. D., Lozano, A. M., Moro, E., Hodaie, M., et al. (2006). Beta oscillatory activity in the subthalamic nucleus and its relation to dopaminergic response in Parkinson's Disease. J. Neurophysiol. 96, 3248-3256. doi: 10.1152/jn.00697.2006.
[2] Ray, N. J., Jenkinson, N., Wang, S., Holland, P., Brittain, J. S., Joint, C., et al. (2008). Local field potential beta activity in the subthalamic nucleus of patients with Parkinson's disease is associated with improvements in bradykinesia after dopamine and deep brain stimulation. Exp. Neurol. 213, 108-113. doi: 10.1016/j.expneurol.2008.05.008
[3] Bronte-Stewart, H., Barberini, C., Koop, M. M., Hill, B. C., Henderson, J. M., and Wingeier, B. (2009). The STN beta-band profile in Parkinson's disease is stationary and shows prolonged attenuation after deep brain stimulation. Exp. Neurol. 215, 20-28. doi: 10.1016/j.expneurol.2008.09.008
[4] Goyal, A., Goetz, S., Stanslaski, S., Oh, Y., Rusheen, A. E., Klassen, B., et al. (2021). The development of an implantable deep brain stimulation device with simultaneous chronic electrophysiological recording and stimulation in humans. Biosen. Bioelectron. 176:112888. doi: 10.1016/j.bios.2020.112888
[5] Feldmann, L. K., Neumann, W.-J., Krause, P., Lofredi, R., Schneider, G.-H., and Kühn, A. A. (2021). Subthalamic beta band suppression reflects effective neuromodulation in chronic recordings. Eur. J. Neurol. 28, 2372-2377. doi: 10.1111/ene.14801

1. A method comprising:
a) receiving, at a processor, first data indicating a value of a local field potential (LFP) over a first time period from one or more first contacts positioned adjacent a region of a brain of a subject;
b) receiving, at the processor, second data indicating a first frequency spectrum of the first data over the first time period;
c) receiving, at the processor, third data indicating a value of a LFP over each of a respective plurality of second time periods from the one or more first contacts, wherein the region of the brain is stimulated by one or more second contacts over the respective plurality of second time periods based on a respective plurality of values of a stimulation parameter;
d) receiving, at the processor, fourth data indicating a value of a plurality of second frequency spectrums of the third data for each of the respective plurality of values of the stimulation parameter;
e) determining, with the processor, a frequency band that encompasses a characteristic of a difference between a value of a parameter of the first frequency spectrum and a value of a parameter of one of the plurality of second frequency spectrums;
f) determining, with the processor, a value of a parameter of each of the plurality of second frequency spectrums over the frequency band for each respective value of the stimulation parameter; and
g) determining, with the processor, a value of an optimal stimulation parameter based on the value of the parameter of each of the plurality of second frequency spectrums over the frequency band and the respective value of the stimulation parameter in step f).

2. The method of claim 1, wherein step g) comprises:
determining, with the processor, a curve that indicates a best fit between the values of the parameter of each of the plurality of second frequency spectrums and the value of the stimulation parameter in step f); and
determining, with the processor, the value of the optimal stimulation parameter based on a value of a feature of the curve.

3. The method of claim 1, wherein the region of the brain is the globus pallidus internus (GPi).

4. The method of claim 2, wherein the one or more second contacts is a plurality of second contacts and wherein steps c) through g) are repeated for each of the plurality of second contacts stimulating the region of the brain over the respective plurality of second time periods based on the respective plurality of values of the stimulation parameter;
and wherein step g) further comprises determining, with the processor, an optimal contact among the plurality of second contacts to stimulate the region of the brain based on comparing the values of the feature of the curve for each of the respective plurality of second contacts.

5. The method of claim 4, wherein the plurality of second contacts comprise:
one or more ring contacts configured to stimulate the region of the brain in multiple directions around a circumference of the ring contact; and
one or more segment contacts configured stimulate the region of the brain in a single direction.

6. The method of claim 1, wherein the stimulation parameter is an amplitude of a signal transmitted by the one or more second contacts to stimulate the region of the brain and wherein the value of the amplitude is varied for each respective plurality of second time periods.

7. The method of claim 1, wherein step e) comprises determining the frequency band that encompasses a maximum value of the difference between the value of the parameter of the first frequency spectrum and the value of the parameter of the second frequency spectrum.

8. The method of claim 7, further comprising observing side effects in the subject during the stimulation by the one or more second contacts over the respective plurality of second time periods and wherein the second frequency spectrum used in step e) corresponds to the second frequency spectrum at a highest value of the stimulation parameter where side effects were not observed.

9. The method of claim 1, wherein the frequency band in step e) includes a beta range of frequency between about 13 Hz and about 30 Hz.

10. The method of claim 1, wherein the value of the parameter of the second frequency spectrums in step f) is at least one of an average value and a range of values of each of the plurality of second frequency spectrums over the frequency band.

11. The method of claim 2, wherein the best fit in step g) comprises a log power fit to generate the curve based on the equation L * e^{-k(x-x0)} +L₀ where x is the value of the stimulation parameter and L, k, x₀, and L₀ are fixed values.

12. The method of claim 2, wherein the best fit in step g) comprises an inverse sigmoid fit to generate the curve based on the equation.
$$\frac{L}{1 + {e}^{- k ( x - {x}_{0} )}} + {L}_{0}$$
where x is the value of the stimulation parameter and L, k, x₀, and L₀ are fixed values.

13. The method of claim 2, wherein the best fit in step g) comprises:
a log power fit to generate the curve based on the equation L * e^{-k(x-x0)} +L₀ where x is the value of the stimulation parameter and L, k, x₀, and L₀ are fixed values; and
an inverse sigmoid fit to generate the curve based on the equation
$$\frac{L}{1 + {e}^{- k ( x - {x}_{0} )}} + {L}_{0}$$
where x is the value of the stimulation parameter and L, k, x₀, and L₀ are fixed values;
wherein step g) further comprises determining a first value indicating a degree that the log power fit curve fits the values of the parameter of each of the second frequency spectrums and determining a second value indicating a degree that the inverse sigmoid fit curve fits the values of the parameter of each of the second frequency spectrums and selecting one of the log power fit curve and the inverse sigmoid fit curve to be used in step h) based on a highest value between the first value and the second value.

14. The method of claim 2, wherein the value of the feature of the curve in step g) is at least one of:
a maximum change in an amplitude of the curve over the values of the stimulation parameter;
a value of the curve corresponding to a highest value of the stimulation parameter before the subject experiences stimulation-induced side effects;
the value of the stimulation parameter at which the value of the curve has reduced by a threshold percentage value;
an available therapeutic window comprising a range of values of the stimulation parameter over which the value of the curve has reduced by the threshold percentage value and the subject does not experience stimulation-induced side effects;
wherein, optionally, the threshold percentage value is at least 90%.

15. (canceled)

16. The method of claim 2, wherein the value of the feature indicates a degree of overlap between the curve and the values of the parameter of the second frequency spectrum.

17. The method of claim 1, wherein the value of the optimal stimulation parameter in step g) is one of a value of an amplitude, a frequency, a pulse width and a recharge phase duration of a stimulation signal to stimulate the region of the brain with the one or more second contacts.

18. An apparatus comprising:
one or more first contacts configured to be positioned adjacent a region of a brain of a subject;
one or more second contacts configured to be positioned adjacent the region of the brain;
at least one processor; and
at least one memory including one or more sequences of instructions,
the at least one memory and the one or more sequences of instructions configured to, with the at least one processor, cause the apparatus to perform at least the following,
a) receive first data indicating a value of a local field potential (LFP) over a first time period from the one or more first contacts;
b) receive second data indicating a first frequency spectrum of the first data over the first time period;
c) receive third data indicating a value of the LFP over each of a respective plurality of second time periods from the one or more first contacts, wherein the region of the brain is stimulated by the one or more second contacts over the respective plurality of second time periods based on a respective plurality of values of a stimulation parameter;
d) receive fourth data indicating a value of the plurality of second frequency spectrums of the third data for each of the respective plurality of values of the stimulation parameter;
e) determine a frequency band that encompasses a characteristic of a difference between a value of a parameter of the first frequency spectrum and a value of a parameter of one of the plurality of second frequency spectrums, wherein, optionally, the frequency band in step e) includes a beta range of frequency between about 13 Hz and about 30 Hz;
f) determine a value of a parameter of each of the plurality of second frequency spectrums over the frequency band for each respective value of the stimulation parameter; and
g) determine a value of an optimal stimulation parameter based on the value of the parameter of each of the plurality of second frequency spectrums and each respective value of the stimulation parameter in step f).

19. The apparatus of claim 18, wherein the one or more second contacts comprise:
one or more ring contacts configured to stimulate the region of the brain in multiple directions around a circumference of the ring contact; and
one or more segment contacts configured stimulate the region of the brain in a single direction,
wherein, optionally, the region of the brain in the globus pallidus internus (GPi).

20. The apparatus of claim 18, further comprising at least one electrode that defines at least one lead and wherein the one or more first contacts and the one or more second contacts define respective non-insulated portions of the at least one lead.

21. (canceled)

22. (canceled)

23. A non-transitory computer-readable medium carrying one or more sequences of instructions, wherein execution of the one or more sequences of instructions by one or more processors causes the one or more processors to perform the steps of:
receive first data indicating a value of a local field potential (LFP) over a first time period from one or more first contacts positioned adjacent a region of a brain of a subject;
receive second data indicating a first frequency spectrum of the first data over the first time period;
receive third data indicating a value of the LFP over each of a respective plurality of second time periods from the one or more first contacts, wherein the region of the brain is stimulated by one or more second contacts over the respective plurality of second time periods based on a respective plurality of values of a stimulation parameter;
receive fourth data indicating a value of a plurality of second frequency spectrums of the third data for each of the respective plurality of values of the stimulation parameter;
determine a frequency band that encompasses a characteristic of a difference between a value of a parameter of the first frequency spectrum and a value of a parameter of one of data indicating a value of a parameter of a frequency spectrum of the value of the LFP over each respective time period;
determine a value of a parameter of each of the plurality of second frequency spectrums over the frequency band for each respective value of the stimulation parameter; and
determine a value of an optimal stimulation parameter based on the value of the parameter of each of the plurality of second frequency spectrums and each respective value of the stimulation parameter.
